(19) United States
(12) Patent Application Publication
(10) Pub. No.: US 2018/0344949 A1
(43) Pub. Date: Dec. 6, 2018

(54) SYSTEM FOR DETECTING AND REMOVING A GAS BUBBLE FROM A VASCULAR INFUSION LINE

(71) Applicant: Clearline MD, LLC, Woburn, MA (US)

(72) Inventors: Ihsan A. Haddad, Ashland, MA (US); Eric Romeo, North Reading, MA (US); Hieu T. Le, Boston, MA (US); Ella Zaslavsky, Marblehead, MA (US)

(21) Appl. No.: 15/997,168

(22) Filed: Jun. 4, 2018

Related U.S. Application Data
(60) Provisional application No. 62/514,079, filed on Jun. 2, 2017.

(51) Int. Cl. A61M 5/36; A61M 5/168; A61M 39/28; A61M 5/172

(52) U.S. Cl. CPC A61M 5/365; A61M 5/16831; A61M 5/16881; A61M 39/281; A61M 2205/128; A61M 2205/3375; A61M 2205/50; A61M 2205/121; A61M 5/172

(57) ABSTRACT

Apparatus for detecting and removing a gas mass from a vascular infusion line, the apparatus comprising: a cartridge, the cartridge comprising: an inlet port; a chamber having a top end, a bottom end and a side wall extending therebetween; an outlet port; a purge port; a first passageway connecting the inlet port to the side wall of the chamber with an upward inclination; a second passageway connecting the bottom end of the chamber to the outlet port; and a third passageway connecting the top end of the chamber to the purge port.

### REFERENCE TO PENDING PRIOR PATENT APPLICATION

This patent application claims benefit of pending prior U.S. Provisional Patent Application Ser. No. 62/514,079, filed Jun. 2, 2017 by Clearline MD, LLC and Ihsan A. Haddad et al. for SYSTEM FOR DETECTING AND REMOVING A GAS BUBBLE FROM A VASCULAR INFUSION LINE (Attorney's Docket No. HAD-21 PROV), which patent application is hereby incorporated herein by reference.

### FIELD OF THE INVENTION

This invention relates to medical apparatus and procedures in general, and more particularly to medical apparatus and procedures for introducing a liquid into the vascular system of a patient.

### BACKGROUND OF THE INVENTION

When a liquid is to be introduced into the vascular system of a patient, it is important to avoid introducing a gas bubble (also known as a “gas volume” or a “gas bolus” or a “gas mass”) into the vascular system of the patient, since such a gas bubble can create a gas embolism in the vascular system of the patient. Such a gas embolism can restrict blood flow through the vascular system of a patient and can result in serious complications, morbidity and even death.

By way of example but not limitation, data suggests that the air in an empty 4 ounce cup, if injected into the vascular system of a 170 pound person, would typically prove lethal if not treated immediately. Such treatment is specialized and may not be available at that point of care. By way of further example but not limitation, for a baby, a gas mass of as little as 0.4 mL can be lethal if not treated immediately. Again, such treatment is specialized and may not be available at that point of care.

It should be appreciated that air can be unintentionally introduced into an infusion line in a variety of circumstances, e.g., when changing infusion bags, when administering fluids by syringe, when using fluid warmers, etc.

Thus there is a need for a system for detecting and removing a gas bubble from a vascular infusion line prior to the gas bubble entering the vascular system of a patient.

### SUMMARY OF THE INVENTION

The present invention comprises the provision and use of a novel system for detecting a gas bubble in a vascular infusion line and removing the gas bubble from the vascular infusion line before the gas bubble can enter the vascular system of a patient.

More particularly, the present invention comprises the provision and use of a novel system which is configured to detect a gas bubble in a vascular infusion line and, upon detecting the gas bubble, divert the flow into a disposable collection bag until the vascular infusion line is free of the gas bubble, at which point the vascular infusion line is again directed into the vascular system of the patient. In essence, the present invention provides a novel system for detecting a gas bubble in a vascular infusion line, entrapping the gas bubble within the system, and purging the entrapped gas bubble before the gas bubble can enter the vascular system of a patient.

In one preferred form of the invention, there is provided a novel system for detecting a gas bubble in a vascular infusion line and removing the gas bubble from the vascular infusion line before the gas bubble can enter the vascular system of a patient, wherein the novel system comprises three components:
(i) a disposable cartridge for disposition intermediate the vascular infusion line, wherein the fluid flowing through the disposable cartridge can be monitored and, if a gas bubble is detected within the fluid flow, the fluid flow containing the gas bubble may be diverted, and the gas bubble removed, before continuing the fluid flow into the patient;
(ii) a base unit for monitoring the fluid flow through the disposable cartridge and, if a gas bubble is detected, diverting the fluid flow containing the gas bubble away from the patient while the gas bubble is removed; and
(iii) a disposable collection bag for receiving the diverted fluid flow containing the gas bubble.

Significantly, the cartridge and the collection bag are the only portions of the novel system which are normally “contaminated” during use (i.e., the cartridge and the collection bag are the only portions of the novel system which are normally contacted by the fluid of the vascular infusion line during use). And significantly, the cartridge and the collection bag are formed out of relatively inexpensive components so that the cartridge and the collection bag can be disposable.

And significantly, the base unit of the novel system, which comprises relatively expensive components such as electronics, sensors, electromechanical units, etc., is not normally “contaminated” during use (i.e., the base unit is not normally contacted by the fluid of the vascular infusion line during use). Thus, the relatively expensive base unit can be configured to be reusable.

In another preferred form of the invention, there is provided apparatus for detecting and removing a gas mass from a vascular infusion line, said apparatus comprising:
a cartridge, said cartridge comprising:
   an inlet port;
   a chamber having a top end, a bottom end and a side wall extending therebetween;
   an outlet port;
   a purge port;
   a first passageway connecting said inlet port to said side wall of said chamber with an upward inclination;
   a second passageway connecting said bottom end of said chamber to said outlet port; and
   a third passageway connecting said top end of said chamber to said purge port.

In another preferred form of the invention, there is provided a method for detecting and removing a gas mass from a vascular infusion line, said method comprising:
providing apparatus comprising:
   a cartridge, said cartridge comprising:
      an inlet port;
      a chamber having a top end, a bottom end and a side wall extending therebetween;
      an outlet port;
      a purge port;
      a first passageway connecting said inlet port to said side wall of said chamber with an upward inclination;
      a second passageway connecting said bottom end of said chamber to said outlet port; and
      a third passageway connecting said top end of said chamber with said purge port;
closing off said second passageway;
introducing a liquid into said inlet port until said liquid exits said purge port;
closing off said third passageway; and
opening up said second passageway so that liquid exits said outlet port.

In another preferred form of the invention, there is provided apparatus for detecting and removing a gas mass from a vascular infusion line, said apparatus comprising:
a cartridge, said cartridge comprising:
   an inlet port;
   a chamber;
   an outlet port;
   a purge port;
   a first passageway connecting said inlet port to said chamber;
   a second passageway connecting said chamber to said outlet port; and
   a third passageway connecting said chamber to said purge port;
a base unit, said base unit comprising a housing and a door movably mounted to said housing between an open position and a closed position, wherein said door is configured to releasably seat said cartridge; and
at least one projection disposed on said door, said at least one projection comprising a concave surface for engaging at least one of said first passageway and said second passageway when a cartridge is seated on said door and said door is in its closed position.

In another preferred form of the invention, there is provided apparatus for detecting and removing a gas mass from a vascular infusion line, said apparatus comprising:
a housing;
a first ultrasound bubble detector mounted to said housing; and
a second ultrasound bubble detector mounted to said housing;
said first ultrasound bubble detector comprising a first channel having an upper end and a lower end, and a first ultrasound transducer disposed adjacent the lower end of said first channel; and
said second ultrasound bubble detector comprising a second channel having an upper end and a lower end, and a second ultrasound transducer disposed adjacent the lower end of said second channel.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1-6 are schematic views showing a novel system for detecting a gas bubble in a vascular infusion line and removing the gas bubble from the vascular infusion line before the gas bubble can enter the vascular system of the patient, wherein the novel system generally comprises a disposable cartridge, a base unit and a disposable collection bag;

FIGS. 7-19 are schematic views showing further details of the disposable cartridge of the novel system shown in FIGS. 1-6;

FIGS. 20-25 are schematic views showing further details of the base unit of the novel system shown in FIGS. 1-6;

FIGS. 26-28 are schematic views showing further details of the disposable collection bag of the novel system shown in FIGS. 1-6;

FIGS. 29 and 30 are schematic views showing alternative components for the base unit of the novel system shown in FIGS. 1-6;

FIGS. 31-34 are schematic views showing alternative components for the base unit of the novel system shown in FIGS. 1-6;

FIGS. 35 and 36 are schematic views showing an alternative approach for mounting the disposable cartridge on the door of the base unit;

FIG. 37 is a schematic view showing an alternative tube holder formed in accordance with the present invention;

FIG. 38 is a schematic view showing an alternative disposable collection bag formed in accordance with the present invention; and

FIGS. 39 and 40 are schematic views showing another disposable cartridge formed in accordance with the present invention.

### DETAILED DESCRIPTION OF THE PREFERRED EMBODIMENTS

The present invention comprises the provision and use of a novel system for detecting a gas bubble in a vascular infusion line and removing the gas bubble from the vascular infusion line before the gas bubble can enter the vascular system of a patient.

More particularly, the present invention comprises the provision and use of a novel system which is configured to detect a gas bubble in a vascular infusion line and, upon detecting the gas bubble, divert the flow into a disposable collection bag until the vascular infusion line is free of the gas bubble, at which point the vascular infusion line is again directed into the vascular system of the patient. In essence, the present invention provides a novel system for detecting a gas bubble in a vascular infusion line, entrapping the gas bubble within the system, and purging the entrapped gas bubble before the gas bubble can enter the vascular system of a patient.

### The Novel System In General

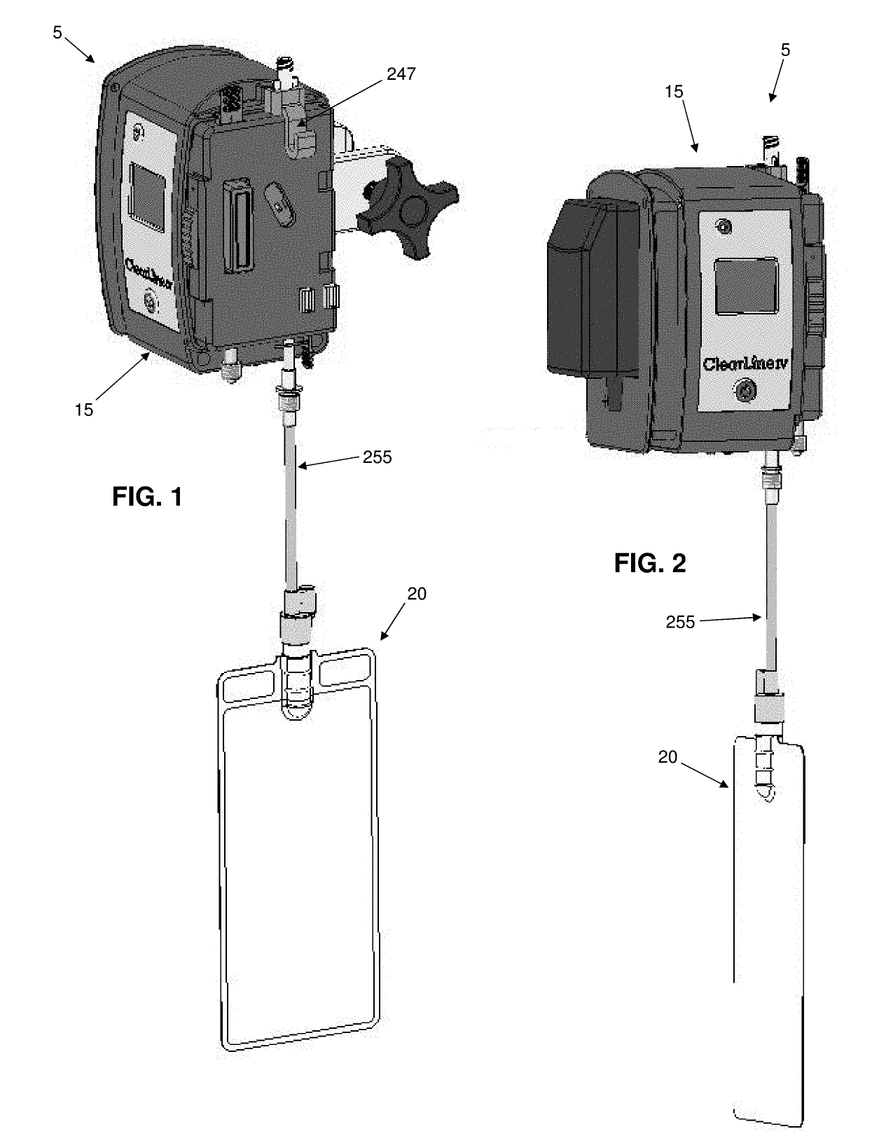

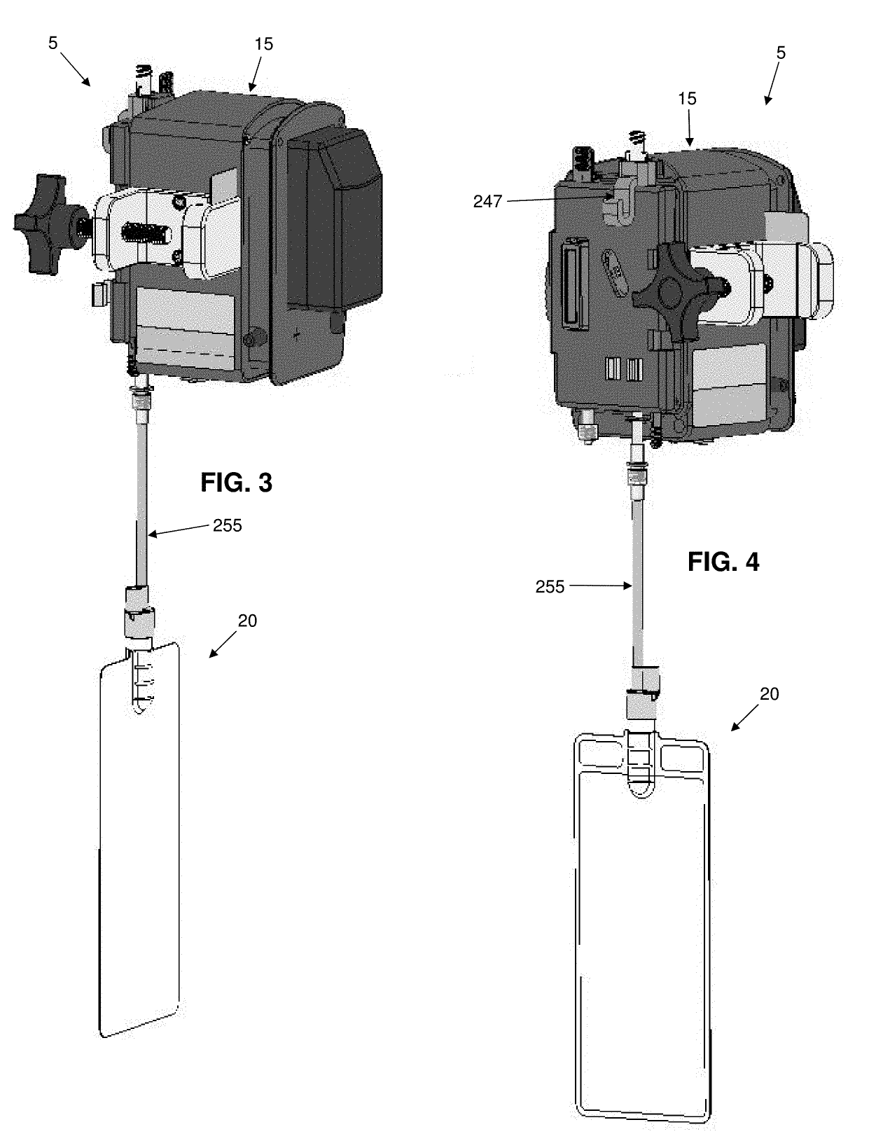

In one preferred form of the invention, and looking now at FIGS. 1-6, there is provided a novel system 5 for detecting a gas bubble in a vascular infusion line and removing the gas bubble from the vascular infusion line before the gas bubble can enter the vascular system of a patient.

Novel system 5 generally comprises three components:
(i) a disposable cartridge 10 for disposition intermediate the vascular infusion line, wherein the fluid flowing through the disposable cartridge can be monitored and, if a gas bubble is detected within the fluid flow, the fluid flow containing the gas bubble may be diverted, and the gas bubble removed, before continuing the fluid flow into the patient;
(ii) a base unit 15 for monitoring the fluid flow through the disposable cartridge and, if a gas bubble is detected, diverting the fluid flow containing the gas bubble away from the patient while the gas bubble is removed; and
(iii) a disposable collection bag 20 for receiving the diverted fluid flow containing the gas bubble.

Significantly, cartridge 10 and collection bag 20 are the only portions of novel system 5 which are normally “contaminated” during use (i.e., cartridge 10 and collection bag 20 are the only portions of novel system 5 which are normally contacted by the fluid of the vascular infusion line during use). And significantly, cartridge 10 and collection bag 20 are formed out of relatively inexpensive components so that cartridge 10 and collection bag 20 can be disposable.

And significantly, base unit 15 of novel system 5, which comprises relatively expensive components such as electronics, sensors, electromechanical units, etc., is not normally “contaminated” during use (i.e., base unit 15 is not normally contacted by the fluid of the vascular infusion line during use). Thus, the relatively expensive base unit 15 can be configured to be reusable.

### The Disposable Cartridge

Disposable cartridge 10 is adapted for disposition intermediate the vascular infusion line, wherein the fluid flowing through the disposable cartridge can be monitored and, if a gas bubble is detected within the fluid flow, the fluid flow containing the gas bubble may be diverted, and the gas bubble removed, before continuing the fluid flow into the patient.

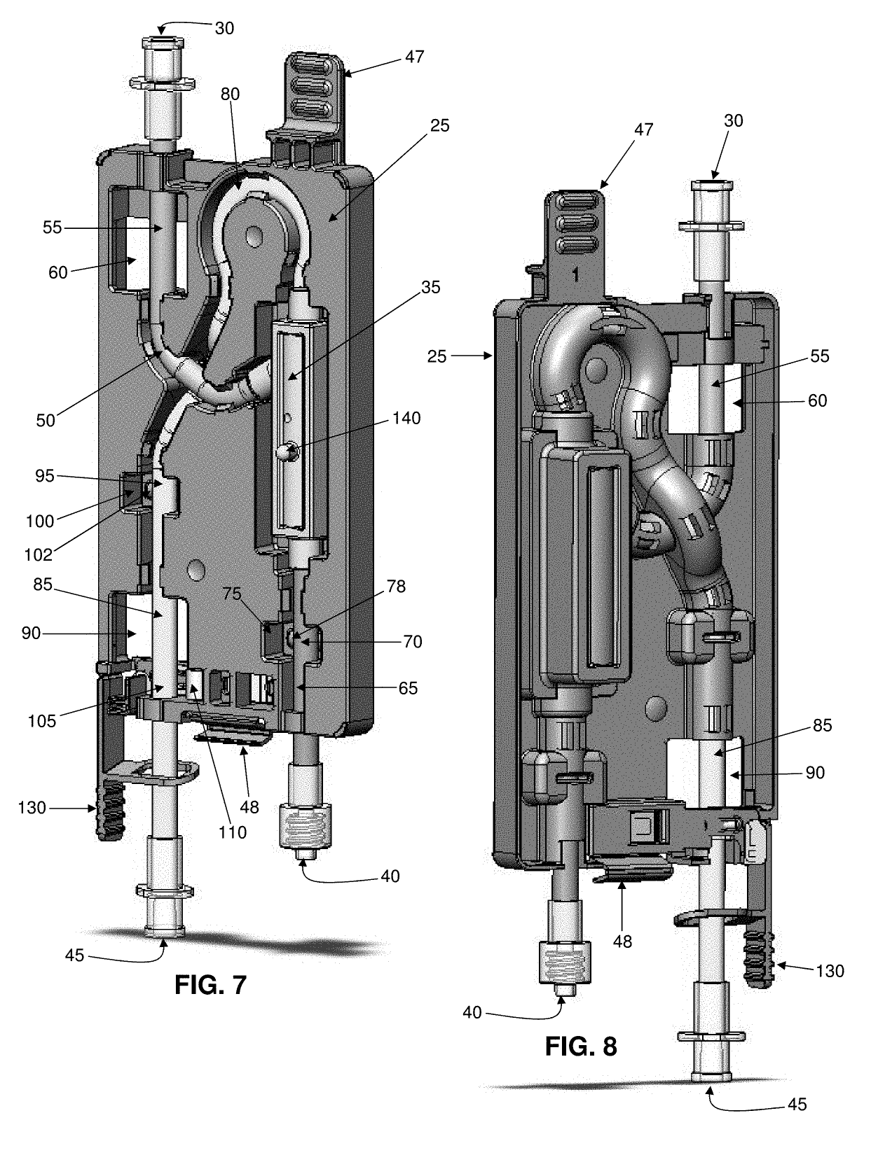

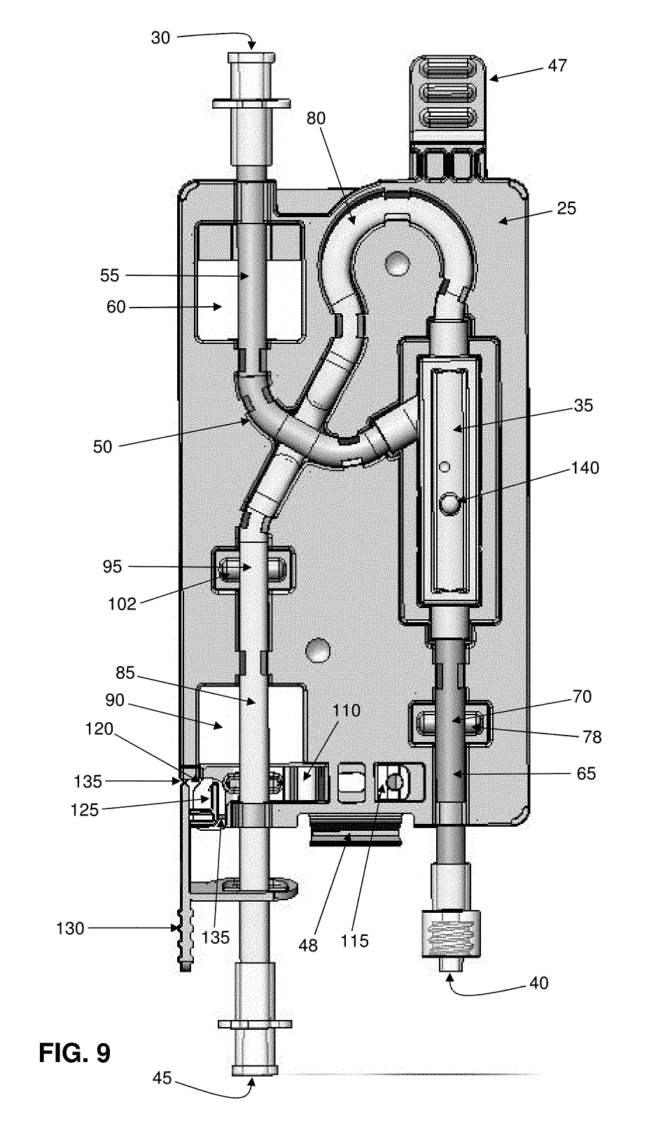

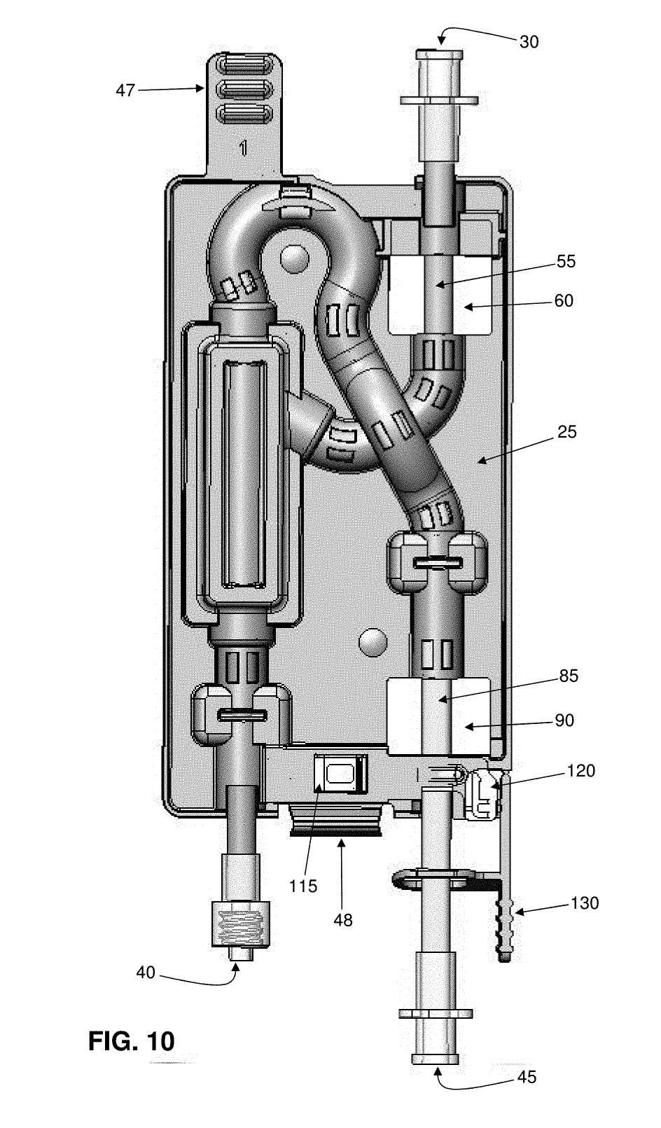

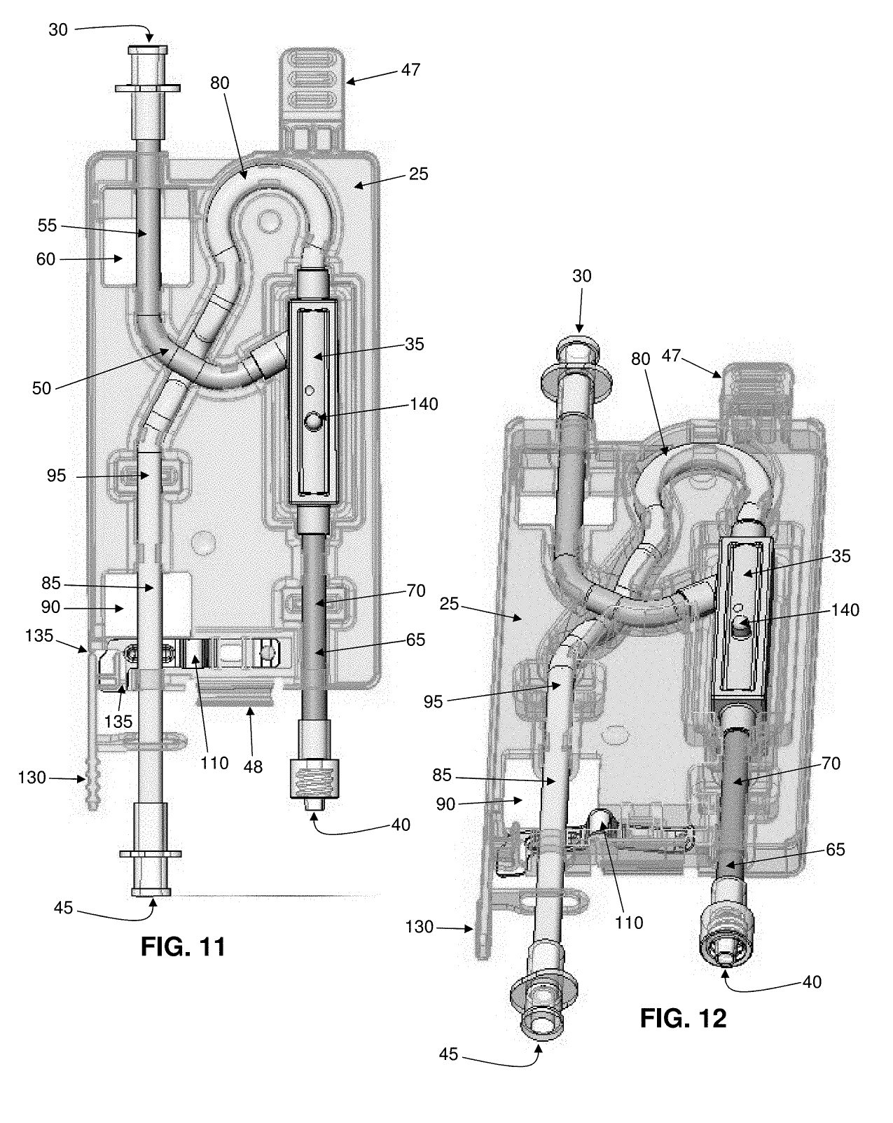

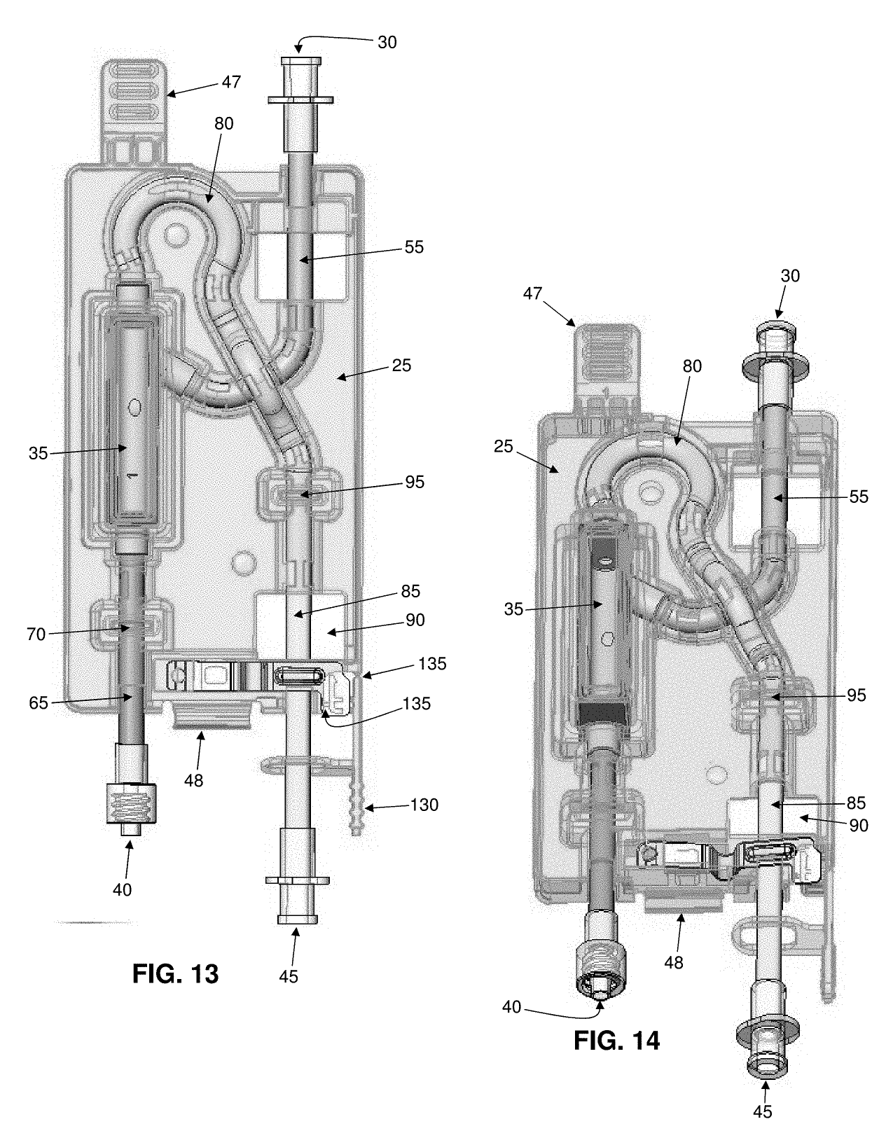

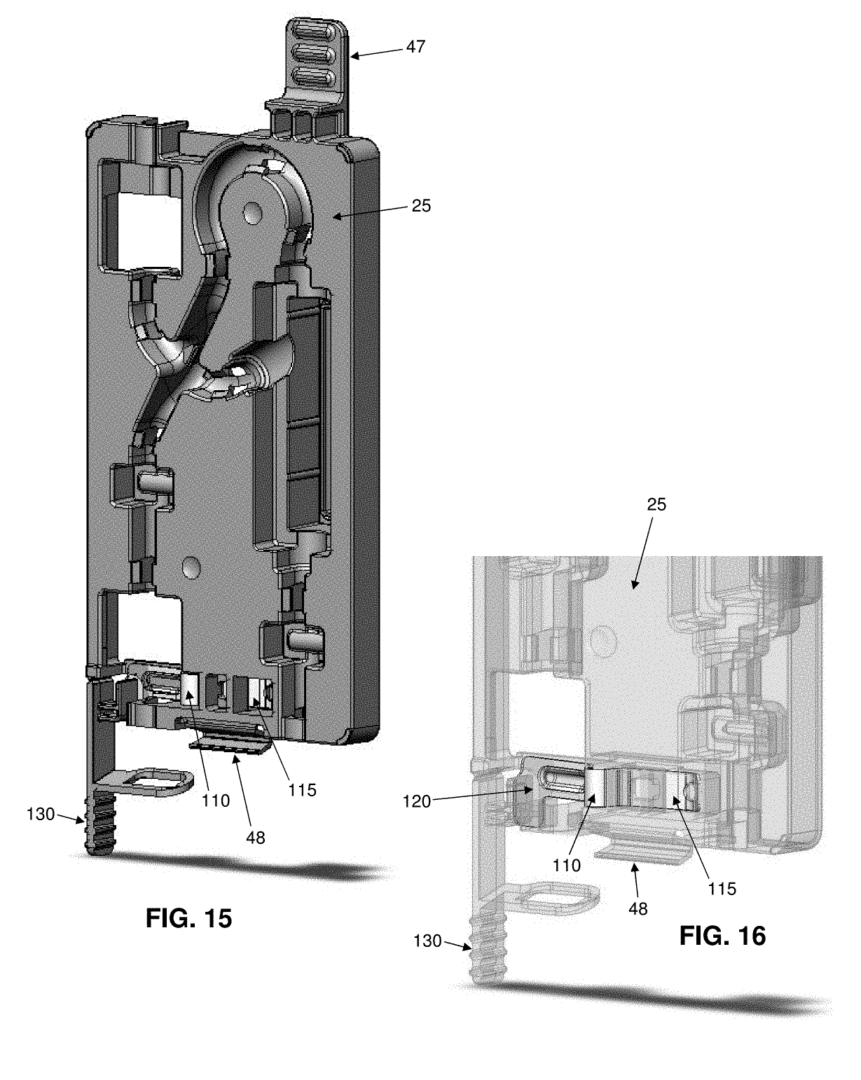

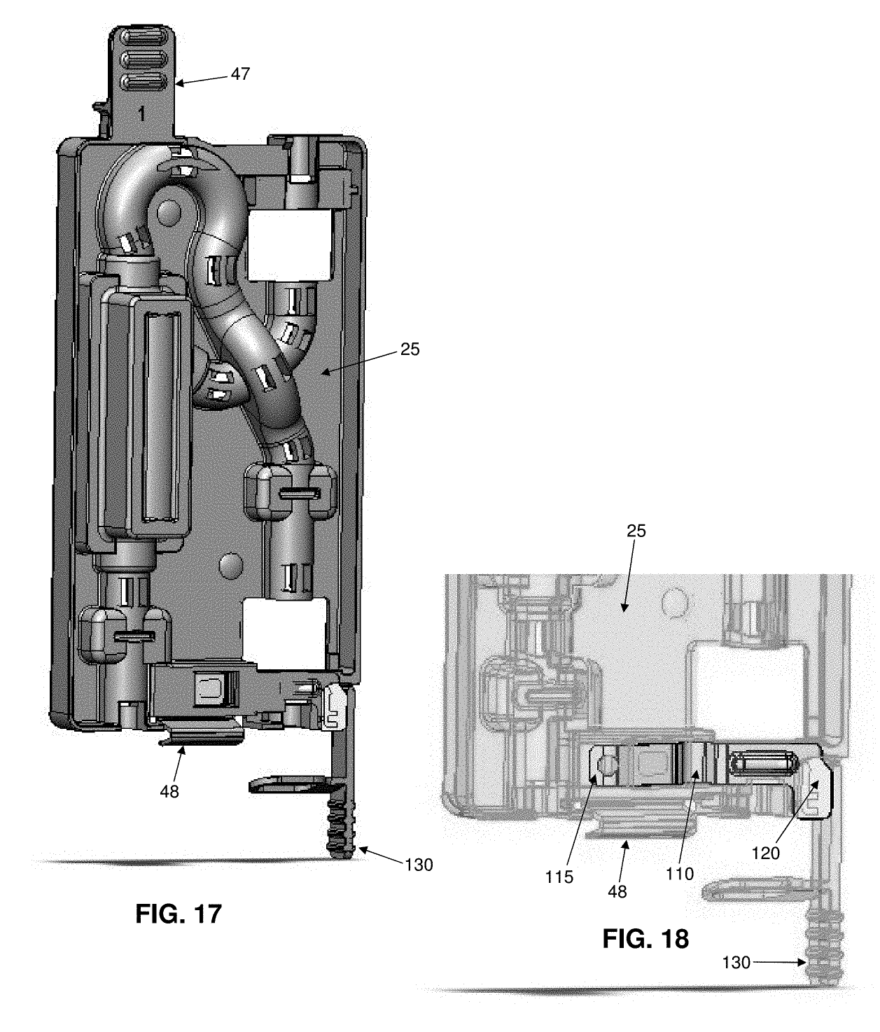

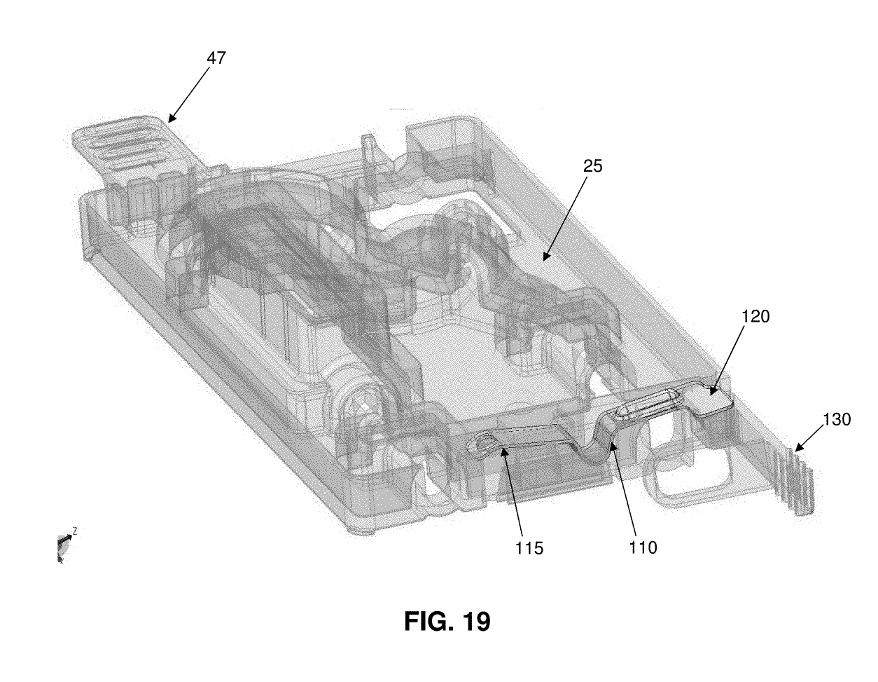

More particularly, and looking now at FIGS. 7-19, disposable cartridge 10 generally comprises a body 25 carrying an inlet port 30, a chamber 35, an outlet port 40, and a purge port 45. As will hereinafter be discussed in further detail, inlet port 30 is intended to be connected to the supply side of an infusion line, chamber 35 is intended to collect microbubbles which are too small for detection by system 5 and allow the collected microbubbles to be purged when a gas bubble large enough to be detected by system 5 is purged (see below), outlet port 40 is intended to be connected to the patient side of an infusion line, and purge port 45 is intended to be connected to disposable collection bag 20. In one preferred form of the invention, a finger tab 47 is provided to facilitate manipulation of disposable cartridge 10 (e.g., to install disposable cartridge 10 on base unit 15, to dismount disposable cartridge 10 from base unit 15, etc.), and a spring tab 48 is provided for releasably securing disposable cartridge 10 on base unit 5 (see below).

A first passageway 50 connects inlet port 30 to chamber 35. First passageway 50 connects to the side wall of chamber 35 near the top end of the side wall of chamber 35, and first passageway 50 preferably enters the side wall of chamber 35 with an upward incline, i.e., so that passageway 50 is “pointing upwards”, towards the top end of chamber 35. First passageway 50 preferably comprises curvatures which are configured to ensure smooth flow with no kinking or restrictions to the fluid flow. First passageway 50 comprises a first bubble detection section 55 where a gas bubble may be detected within first passageway 50. In one preferred form of the invention, first passageway 50 comprises a tube. A first opening 60 (e.g., a hole) is preferably formed in body 25 adjacent to first bubble detection section 55 so that the entire periphery of first bubble detection section 55 is readily accessible.

A second passageway 65 connects chamber 35 with outlet port 40. A first pinch valve section 70 is disposed along second passageway 65 intermediate chamber 35 and outlet port 40, such that first pinch valve section 70 can be selectively closed off to fluid flow, in order that second passageway 65 can be selectively closed off to fluid flow. In one preferred form of the invention, second passageway 65 comprises a tube, and first pinch valve section 70 comprises a section of the tube having a relatively soft side wall which may be easily compressed so as to collapse the tube and close off the tube to fluid flow. A second opening 75 (e.g., a recess) is preferably formed in body 25 adjacent to first pinch valve section 70 so that one side of first pinch valve section 70 is readily accessible, and a protrusion 78 is preferably formed on the base of second opening 75, i.e., on the rear side of first pinch valve section 70.

A third passageway 80 connects chamber 35 with purge port 45. Third passageway 80 connects to the top end of chamber 35. Third passageway 80 preferably comprises curvatures which are configured to ensure smooth flow with no kinking or restrictions to fluid flow. Third passageway 80 comprises a second bubble detection section 85 where a gas bubble may be detected within third passageway 80. In one preferred form of the invention, third passageway 80 comprises a tube. A third opening 90 (e.g., a hole) is preferably formed in body 25 adjacent to second bubble detection section 85 so that the entire periphery of second bubble detection section 85 is readily accessible. A second pinch valve section 95 is disposed along third passageway 80 intermediate chamber 35 and second bubble detection section 85, such that second pinch valve section 95 can be selectively closed off to fluid flow, in order that third passageway 80 can be selectively closed off to fluid flow. In one preferred form of the invention, second pinch valve section 95 comprises a section of the tube having a relatively soft side wall which may be easily compressed so as to collapse the tube and close off the tube to fluid flow. And in one preferred form of the invention, a fourth opening 100 (e.g., a recess) is formed in body 25 adjacent to second pinch valve section 95 so that one side of pinch valve section 95 is readily accessible, and a protrusion 102 is preferably formed at the base of fourth opening 100, i.e., on the rear side of second pinch valve section 95.

In the preferred form of the invention, a third pinch valve section 105 is disposed along third passageway 80 intermediate second bubble detection section 85 and purge port 45, such that third pinch valve section 105 can be selectively closed off to fluid flow, in order that third passageway 80 can be selectively closed off to fluid flow. In one preferred form of the invention, third pinch valve section 105 comprises a section of the tube having a relatively soft side wall which may be easily compressed so as to collapse the tube and close off the tube to fluid flow.

In the preferred form of the invention, a resilient valve element 110 is secured to body 25 adjacent to third pinch valve section 105. Resilient valve element 110 is configured so that in its unbiased state, resilient valve element 110 closes off third pinch valve section 105 to fluid flow. In one preferred form of the invention, resilient valve element 110 comprises a piece of spring steel having a first end 115 fixed to body 25 and a second end 120 cantilevered across third pinch valve section 105; in its unbiased state, second end 120 of resilient valve element 110 engages third pinch valve section 105 and collapses third pinch valve section 105, forcing third pinch valve section 105 closed.

A removable mechanical stop 125 is provided to selectively hold resilient valve element 110 spaced from third pinch valve section 105, such that third pinch valve section 105 is open to fluid flow. Removable mechanical stop 125 is constructed so that the removable mechanical stop can be selectively withdrawn from resilient valve element 110, such that resilient valve element 110 will engage third pinch valve section 105 and collapse third pinch valve section 105, forcing third pinch valve section 105 closed. In one preferred form of the invention, removable mechanical stop 125 is formed as part of body 25; when removable mechanical stop 125 is to be withdrawn from engagement with resilient valve element 110, a finger tab 130 is used to break removable mechanical stop 125 away from body 25 at a pair of necks 135, thereby freeing resilient valve element 110 to engage third pinch valve section 105 and collapse third pinch valve section 105, forcing third pinch valve section 105 closed.

Disposable cartridge 10 also comprises a protrusion 140 extending out of the plane of the disposable cartridge. In one preferred form of the invention, protrusion 140 is formed as part of chamber 35, i.e., protrusion 140 extends out of the outer surface of chamber 35.

And in one preferred form of the invention, body 25 is molded as a single piece of material having recesses formed therein for receiving chamber 35, first passageway 50, second passageway 65 and third passageway 80, with chamber 35, first passageway 50, second passageway 65, third passageway 80 and resilient valve element 110 being mounted to body 25 during assembly.

It will be appreciated that, in view of the foregoing construction, disposable cartridge 10 essentially comprises an inlet port 30; a chamber 35; an outlet port 40; a purge port 45; a first passageway 50 connecting inlet port 30 to chamber 35; a second passageway 65 connecting chamber 35 with outlet port 40; a third passageway 80 connecting chamber 35 with purge port 45; a first bubble detection section 55 disposed in first passageway 50 intermediate inlet port 30 and chamber 35; a second bubble detection section 85 disposed in third passageway 80 intermediate chamber 35 and purge port 45; a first pinch valve section 70 disposed in second passageway 65 intermediate chamber 35 and outlet port 40; a second pinch valve section 95 disposed in third passageway 80 intermediate chamber 35 and purge port 45; a third pinch valve section 105 disposed in third passageway 80 between second pinch valve section 95 and purge port 45; a resilient valve element 110 mounted to body 25 and configured so that in its unbiased state, resilient valve element 110 will close off third pinch valve section 105; and a removable mechanical stop 125 releasably mounted to body 25 which, when in position on body 25, keeps resilient valve element 110 from closing off third pinch valve section 105.

Significantly, first passageway 50 (connected to the supply side of the infusion line) connects to the side wall of chamber 35 near the top end of the side wall of chamber 35, second passageway 65 (connected to the patient side of the infusion line) connects to the bottom end of chamber 35, and third passageway 80 (connected to the purge port) connects to the top end of chamber 35. As a result of this construction, inasmuch as a gas tends to rise in a liquid, any gas masses contained in the liquid entering chamber 35 from first passageway 50, and any microbubbles entrained in the liquid entering chamber 35 from first passageway 50, will be inclined to rise to the top of chamber 35 and pass out of chamber 35 into the adjacent uppermost portions of third passageway 80, ready for purging through third passageway 80 and purge port 45 when gas masses detected by system 5 are purged through third passageway 80 and purge port 45. At the same time, any sizeable gas masses contained in the liquid entering chamber 35 from first passageway 50, and any microbubbles entrained in the liquid entering chamber 35 from first passageway 50, will be disinclined to descend within chamber 35 and pass out second passageway 65 (connected to the patient side of the infusion line).

Furthermore, inasmuch as first passageway 50 enters the side wall of chamber 35 with an upward incline, i.e., so that first passageway 50 is “pointing upwards”, towards the top end of chamber 35, fluid entering inlet port 30 and traveling through first passageway 50 to chamber 35 enters near the top end of chamber 35, oriented towards the top end of the chamber, so that any sizeable gas masses contained in the liquid, and any microbubbles entrained in the liquid are pre-directed to the top end of chamber 35, where they can collect and then pass into the adjacent uppermost portions of third passageway 80, ready for purging through third passageway 80 and purge port 45 when gas masses detected by system 5 are purged through third passageway 80 and purge port 45.

### The Base Unit

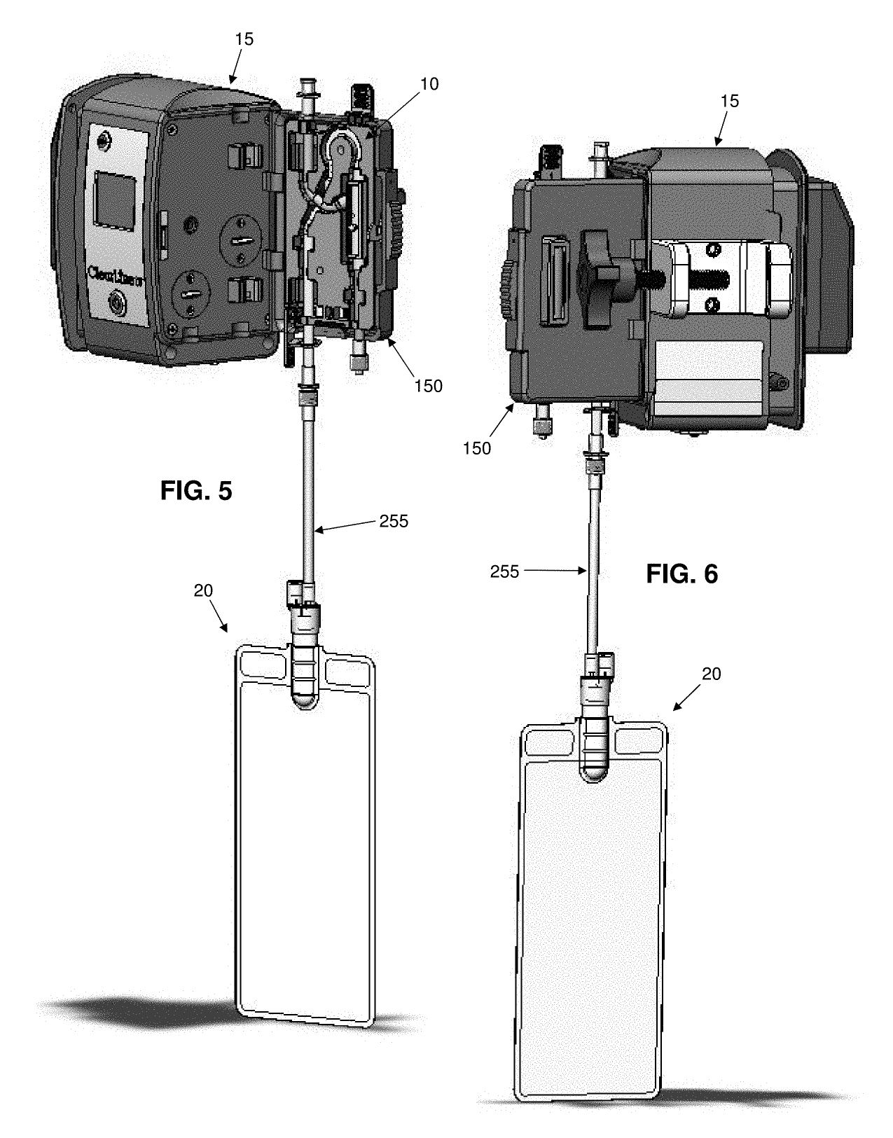

Base unit 15 is configured to receive disposable cartridge 10 and to hold disposable cartridge 10 with a vertical disposition (e.g., with a disposition such as that shown in FIG. 5), and base unit 15 is configured to monitor the fluid flow through disposable cartridge 10 and, if a gas bubble is detected, divert the fluid flow containing the gas bubble away from the patient while the gas bubble is removed.

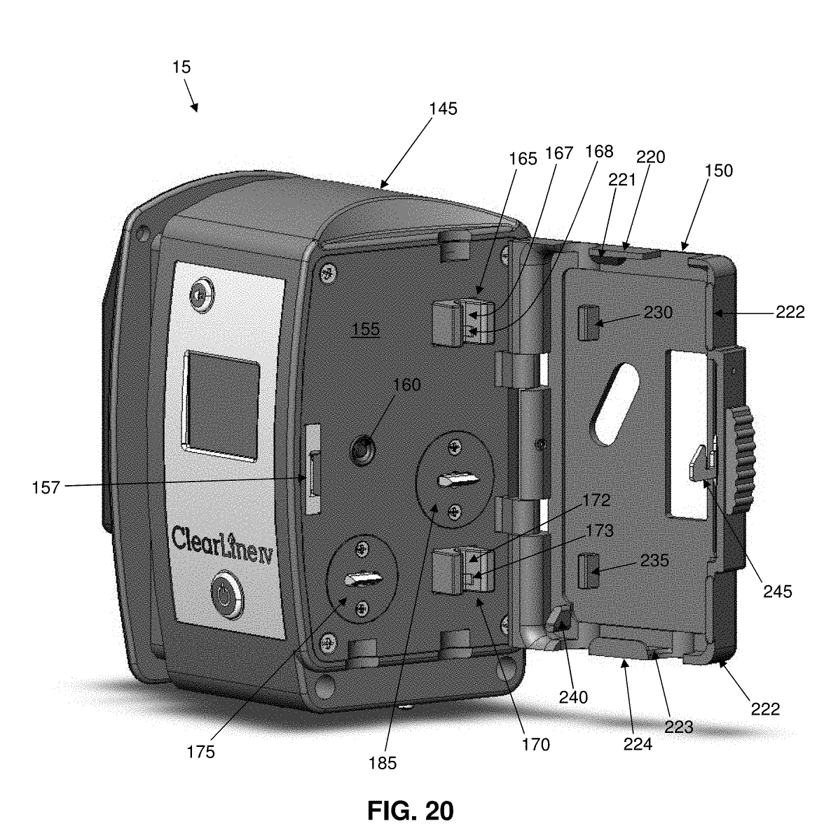

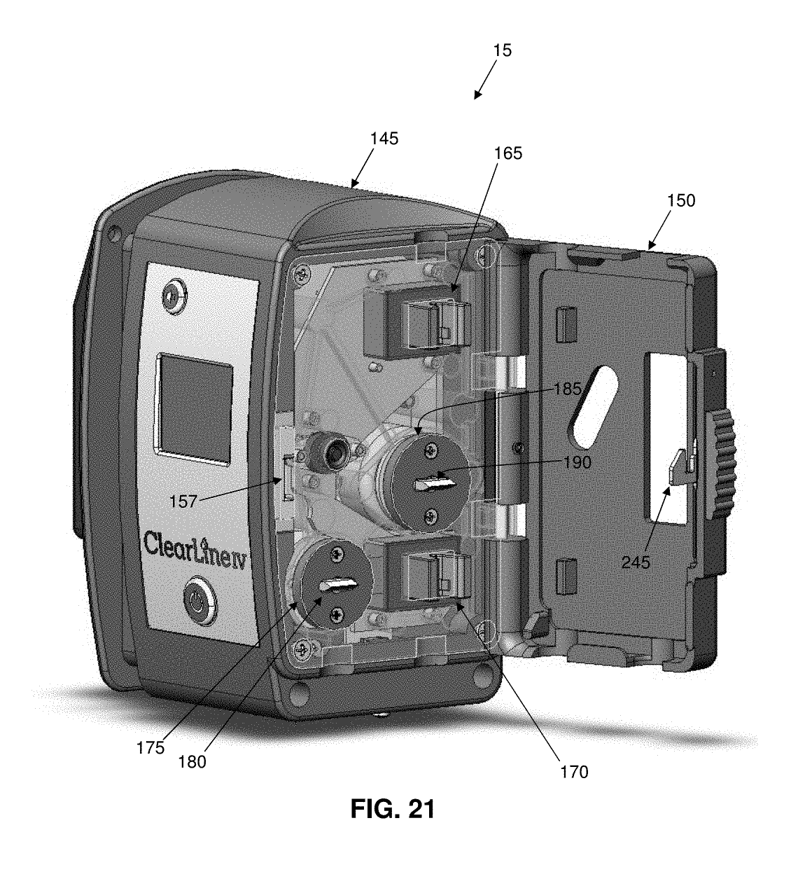

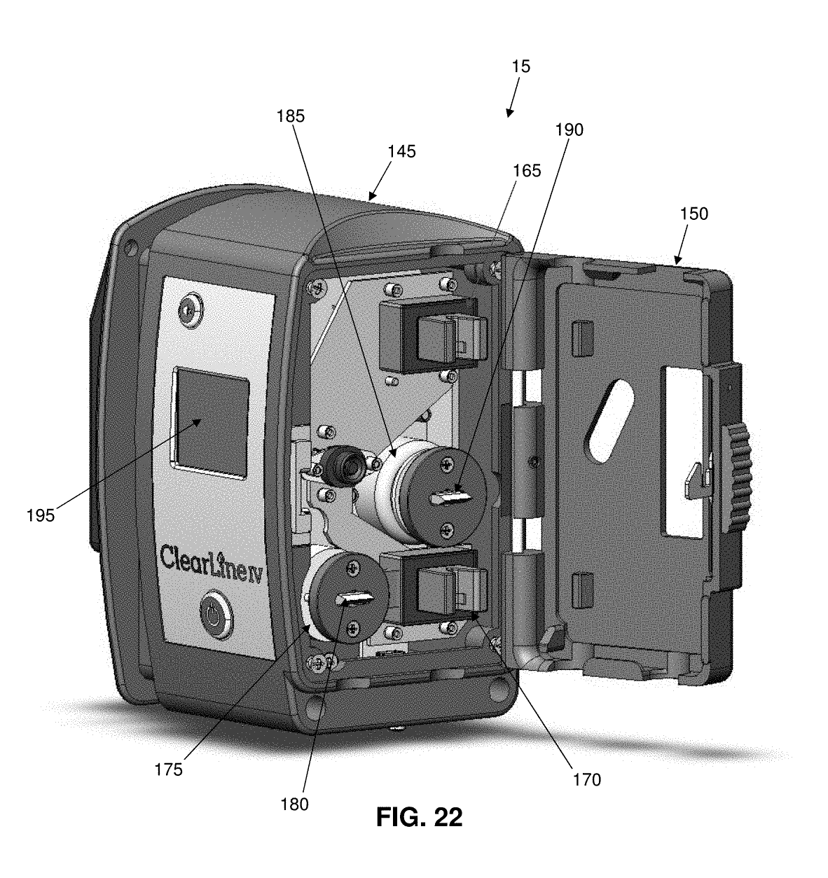

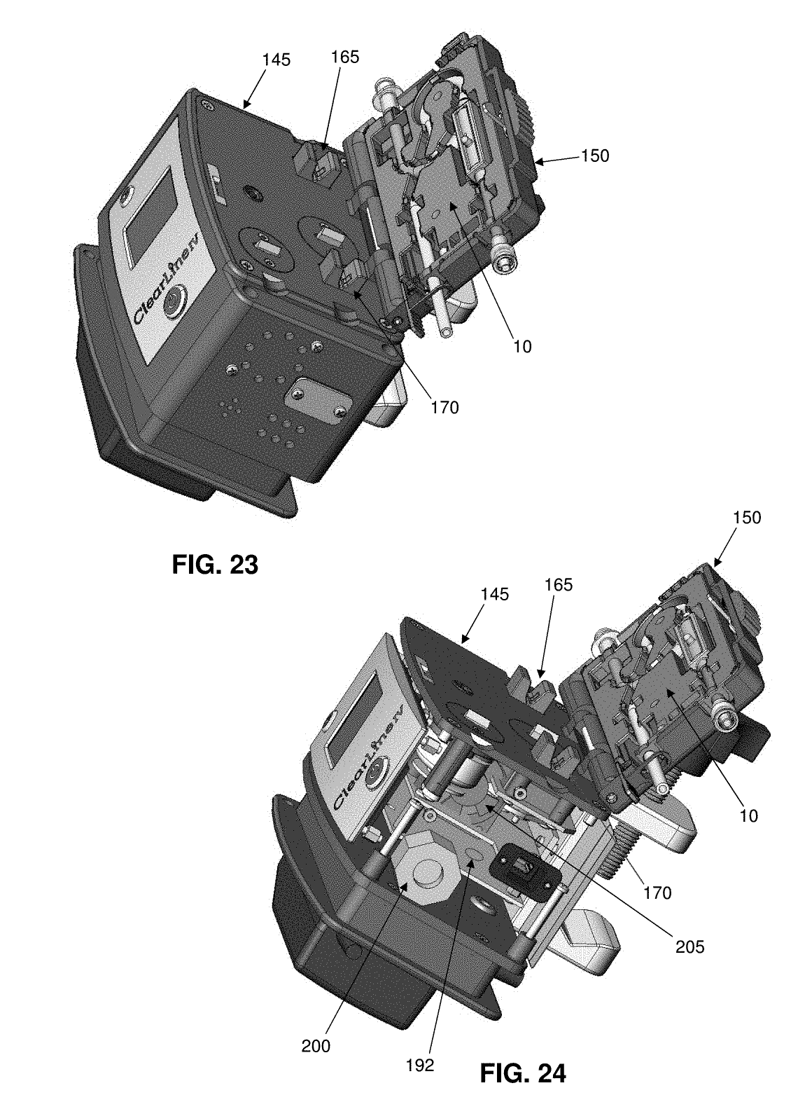

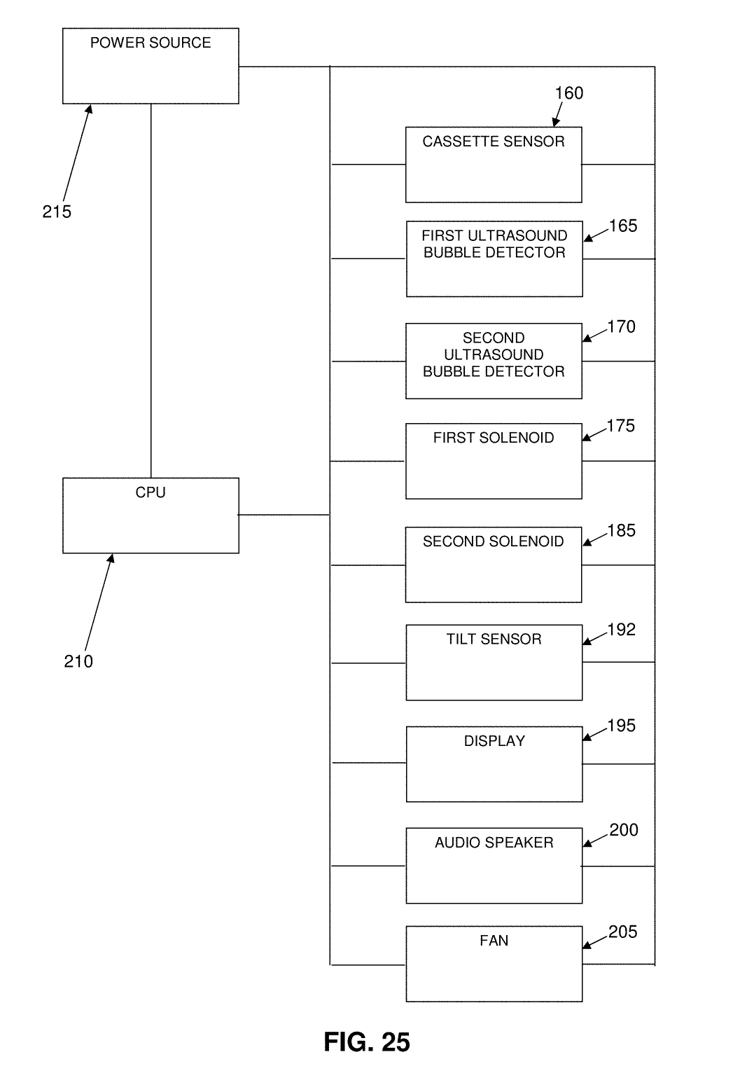

More particularly, looking now at FIGS. 20-25, base unit 15 generally comprises a housing 145 and a door 150.

Housing 145 generally comprises a face 155; a latch seat 157; a cartridge sensor 160 opening on face 155 for determining when a disposable cartridge 10 is mounted to base unit 15; a first ultrasound bubble detector 165 for detecting when a gas bubble is disposed in first bubble detection section 55 of disposable cartridge 10, wherein first ultrasound bubble detector 165 comprises a first channel 167 for receiving first bubble detection section 55 of disposable cartridge 10 and a first ultrasound transducer 168 for detecting a gas bubble disposed in first bubble detection section 55 of disposable cartridge 10; a second ultrasound bubble detector 170 for detecting when a gas bubble is disposed in second bubble detection section 85 of disposable cartridge 10, wherein second ultrasound bubble detector 170 comprises a second channel 172 for receiving second bubble detection section 85 of disposable cartridge 10 and a second ultrasound transducer 173 for detecting a gas bubble disposed in second bubble detection section 85 of disposable cartridge 10; a first solenoid 175 for selectively moving a first pinch valve head 180 into and out of engagement with first pinch valve section 70 of disposable cartridge 10; and a second solenoid 185 for selectively moving a second pinch valve head 190 into and out of engagement with second pinch valve section 95 of disposable cartridge 10.

Note that while first ultrasound transducer 168 can detect a gas bubble disposed in first bubble detection section 55 of disposable cartridge 10, first ultrasound transducer 168 can also detect a liquid disposed in first bubble detection section 55 of disposable cartridge 10. Note also that while second ultrasound transducer 173 can detect a gas bubble disposed in second bubble detection section 85 of disposable cartridge 10, second ultrasound transducer 173 can also detect a liquid disposed in second bubble detection section 85 of disposable cartridge 10.

In one preferred form of the invention, housing 145 also comprises a tilt sensor 192 for detecting excessive tilt of housing 145 (e.g., such as if base unit 15 should fall over); a display 195 for visually displaying system information (e.g., system status, flow rate, etc.) and warnings to a user; an audio speaker 200 for audibly presenting system information and warnings to a user; a fan 205 for cooling various housing components; a central processing unit (CPU) 210 which is appropriately programmed in ways well known in the art so as to provide system functionality as will be hereinafter discussed; and a power source 215 (e.g., a battery carried by housing 145, an AC adapter which plugs into housing 145, etc.) for powering the various system components.

In one preferred form of the invention, first ultrasound bubble detector 165 and second ultrasound bubble detector 170 are configured to detect gas masses having a volume of 25 microliters or larger, so that such gas masses can be purged from the fluid line by system 5. In one particularly preferred form of the invention, first ultrasound bubble detector 165 and second ultrasound bubble detector 170 are configured to detect gas masses having a volume of 6 microliters or larger, so that such gas masses can be purged from the fluid line by system 5. Note also that microbubbles having a volume smaller than the detection threshold of first ultrasound bubble detector 165 and second ultrasound bubble detector 170 will collect at the top of chamber 35 and then pass into the adjacent uppermost portions of third passageway 80, ready for purging through third passageway 80 and purge port 45 when gas masses detected by first ultrasound bubble detector 165 and second ultrasound bubble detector 170 are purged through third passageway 80 and purge port 45.

In one preferred form of the invention, first ultrasound transducer 168 is disposed in the lower portion of first channel 167 of first ultrasound bubble detector 165 (i.e., adjacent to the egress of first bubble detection section 55 from first channel 167), such that first ultrasound transducer 168 is spaced from the point where first bubble detection section 55 of disposable cartridge 10 enters first channel 167 of first ultrasound bubble detector 165 (i.e., spaced from the ingress of first bubble detection section 55 into first channel 167). More particularly, it has been found that when first bubble detection section 55 of disposable cartridge 10 is advanced into first channel 167 of first ultrasound bubble detector 165 (see below), first bubble detection section 55 may be squeezed inwardly slightly, creating a narrowing of the lumen of first bubble detection section 55, and this narrowing may cause a gas bubble to lodge in the line just proximal to the narrowing, particularly when the flow rate of the infusion line is relatively low. In this event, if first ultrasound transducer 168 is located in the upper portion of first channel 167 of first ultrasound bubble detector 165, first ultrasound transducer 168 could report the continuous presence of a gas bubble at first ultrasound bubble detector 165, thereby causing system 5 to continuously purge the infusion fluid even though the infusion fluid may be substantially devoid of gas masses. Therefore, to eliminate this possibility, first ultrasound transducer 168 is preferably located in the lower portion of first channel 167 of first ultrasound bubble detector 165, adjacent to the egress of first bubble detection section 55 from first channel 167. By way of example but not limitation, where first channel 167 of first ultrasound bubble detector 165 has a length of approximately 0.460 inch, first ultrasound transducer 168 may be positioned approximately 0.325 inch from the top end of first channel 167 (e.g., approximately 70.6% of the way down the length of first channel 167).

And in one preferred form of the invention, second ultrasound transducer 173 is disposed in the lower portion of second channel 172 of second ultrasound bubble detector 170 (i.e., adjacent to the egress of second bubble detection section 85 from second channel 172), such that second ultrasound transducer 173 is spaced from the point where second bubble detection section 85 of disposable cartridge 10 enters second channel 172 of second ultrasound bubble detector 170 (i.e., spaced from the ingress of second bubble detection section 85 into second channel 172). More particularly, it has been found that when second bubble detection section 85 of disposable cartridge 10 is advanced into second channel 172 of second ultrasound bubble detector 170 (see below), second bubble detection section 85 may be squeezed inwardly slightly, creating a narrowing of the lumen of second bubble detection section 85, and this narrowing may cause a gas bubble to lodge in the line just proximal to the narrowing, particularly when the flow rate of the infusion line is relatively low. In this event, if second ultrasound transducer 173 is located in the upper portion of second channel 172 of second ultrasound bubble detector 170, second ultrasound transducer 173 could report the continuous presence of a gas bubble at second ultrasound bubble detector 170, thereby causing system 5 to continuously purge the infusion fluid even though the infusion fluid may be substantially devoid of gas masses. Therefore, to eliminate this possibility, second ultrasound transducer 173 is preferably located in the lower portion of second channel 172 of second ultrasound bubble detector 170, adjacent to the egress of second bubble detection section 85 from second channel 172. By way of example but not limitation, where second channel 172 of second ultrasound bubble detector 170 has a length of approximately 0.460 inch, second ultrasound transducer 173 may be positioned approximately 0.325 inch from the top end of second channel 172 (e.g., approximately 70.6% of the way down the length of second channel 172).

Door 150 is configured to releasably seat disposable cartridge 10 thereon (e.g., disposable cartridge 10 is releasably captured on door 150 via (i) first door edge 220 and its associated flange 221, a second door edge 222, and a third door edge 223 and its associated flange 224, and (ii) spring tab 48 on disposable cartridge 10), with a first finger 230 of door 150 engaging first bubble detection 55 of disposable cartridge 10, a second finger 235 of door 150 engaging second bubble detection section 85 of disposable cartridge 10, and with a third finger 240 of door 150 engaging second end 120 of resilient valve element 110 (i.e., whereby to hold second end 120 of resilient valve element 110 away from third pinch valve section 105 of third passageway 80 when removable mechanical stop 125 has been removed from body 25). In one preferred form of the invention, disposable cartridge 10 is releasably mounted to door 150 by positioning spring tab 48 of disposable cartridge 10 on third door edge 223, behind its associated flange 224; pushing downward on disposable cartridge 10 so as to compress spring tab 48 on disposable cartridge 10; pushing the top end of disposable cartridge 10 toward door 150 so that the top end of disposable cartridge 10 clears flange 221 on first door edge 220; and then releasing disposable cartridge 10 so that spring tab 48 on disposable cartridge 10 pushes the top end of disposable cartridge 10 up behind flange 221 of first door edge 220.

Door 150 is hingedly mounted to housing 145 so that when door 150 is closed and latched shut (e.g., by means of latch finger 245 of door 150 engaging latch seat 157 of housing 145), a disposable cartridge 10 seated on door 150 is brought into engagement with face 155 of housing 145. More particularly, when a disposable cartridge 10 is seated on door 150 and door 150 is thereafter closed:
(i) protrusion 140 of disposable cartridge 10 engages cartridge sensor 160 on housing 145, whereby to advise CPU 210 that a disposable cartridge 10 is mounted to door 150 of base unit 15 and that door 150 is closed and latched;
(ii) first bubble detection section 55 of disposable cartridge 10 is seated in first ultrasound bubble detector 165 on housing 145, with first finger 230 of door 150 ensuring that first bubble detection section 55 of disposable cartridge 10 is securely seated in first channel 167 of first ultrasound bubble detector 165 of housing 145, with first ultrasound transducer 168 of first ultrasound bubble detector 165 disposed adjacent to first bubble detection section 55, such that first ultrasound bubble detector 165 can advise CPU 210 when a gas bubble is disposed in first bubble detection section 55 of disposable cartridge 10;
(iii) second bubble detection section 85 of disposable cartridge 10 is seated in second ultrasound bubble detector 170 on housing 145, with second finger 235 of door 150 ensuring that second bubble detection section 85 is securely seated in second channel 172 of second ultrasound bubble detector 170 of housing 145, with second ultrasound transducer 173 of second ultrasound bubble detector 170 disposed adjacent to second bubble detection section 85, such that second ultrasound bubble detector 170 can advise CPU 210 when a gas bubble is disposed in second bubble detection section 85 of disposable cartridge 10;
(iv) first pinch valve head 180 of first solenoid 175 is aligned with first pinch valve section 70 of disposable cartridge 10, such that first pinch valve section 70 of disposable cartridge 10 can be closed off to fluid flow when first solenoid 175 causes first pinch valve head 180 to be driven against first pinch valve section 70, whereby to collapse first pinch valve section 70 between first pinch valve head 180 and protrusion 78 of second opening 75 of body 25; and
(v) second pinch valve head 190 of second solenoid 185 is aligned with second pinch valve section 95 of disposable cartridge 10, such that second pinch valve section 95 of disposable cartridge 10 can be closed off to fluid flow when second solenoid 185 causes second pinch valve head 190 to be driven against second pinch valve section 95, whereby to collapse second pinch valve section 95 between second pinch valve head 190 and protrusion 102 of fourth opening 100 of body 25.

First solenoid 175 is preferably a so-called “spring-return” solenoid, and first solenoid 175 is preferably disposed in housing 145, such that first pinch valve head 180 of first solenoid 175 is normally biased “inwardly” (i.e., away from a disposable cartridge 10 mounted to door 150), and the application of an electric current to first solenoid 175 causes first pinch valve head 180 of first solenoid 175 to be advanced “outwardly” (i.e., toward a disposable cartridge 10 mounted to door 150); and second solenoid 185 is preferably a so-called “spring-return” solenoid, and second solenoid 185 is preferably disposed in housing 145, such that second pinch valve head 190 of second solenoid 185 is normally biased “outwardly” (i.e., toward a disposable cartridge 10 mounted to door 150), and the application of an electric current to second solenoid 185 causes second pinch valve head 190 of second solenoid 185 to retract “inwardly” (i.e., away from a disposable cartridge 10 mounted to door 150). It will be appreciated that, inasmuch as first solenoid 175 and second solenoid 185 have the foregoing constructions and dispositions, the “default” condition of system 5 is for first pinch valve section 70 of disposable cartridge 10 to be open to fluid flow, and for second pinch valve section 95 of disposable cartridge 10 to be closed to fluid flow, when a disposable cartridge 10 is mounted to door 150 and door 150 is closed and latched shut. In other words, when no power is applied to solenoids 175, 185, inlet port 30 is connected to outlet port 40, and inlet port 30 is isolated from purge port 45.

Alternatively, if desired, first solenoid 175 can be a so-called “floating” solenoid which lacks a spring return—in this case, the natural resilience of first pinch valve section 70 can be used to push first pinch valve head 180 toward first solenoid 175, such that first pinch valve section 70 is open to flow. This construction can be advantageous since (i) it allows a less expensive solenoid to be used for first solenoid 175, and (ii) more closure force can be generated by first solenoid 175, inasmuch as first solenoid 175 does not need to fight the power of a spring to advance first pinch valve head 180 against first pinch valve section 70.

If desired, door 150 may also comprise a tube holder/hook 247. More particularly, the tube holder portion of tube holder/hook 247 can help hold the supply side of an infusion line upright where the supply side of the infusion line joins inlet port 30 of disposable cartridge 10, and the hook portion of tube holder/hook 247 can help support excess infusion line tubing adjacent to base unit 15.

### Disposable Collection Bag

Disposable collection bag 20 is configured to be connected to purge port 45 of disposable cartridge 10 and receive the diverted fluid flow containing a gas bubble.

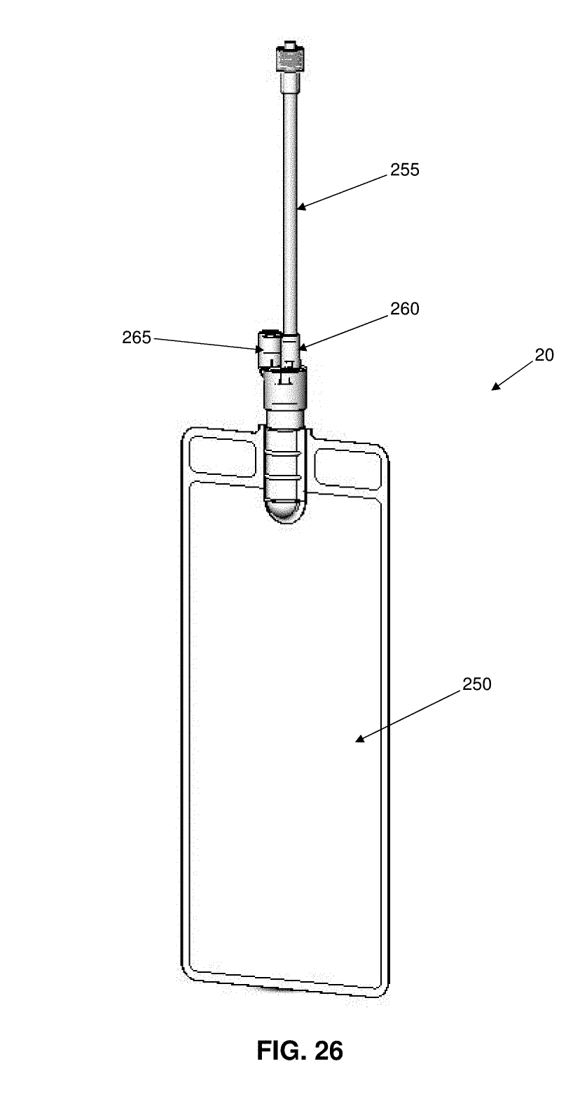

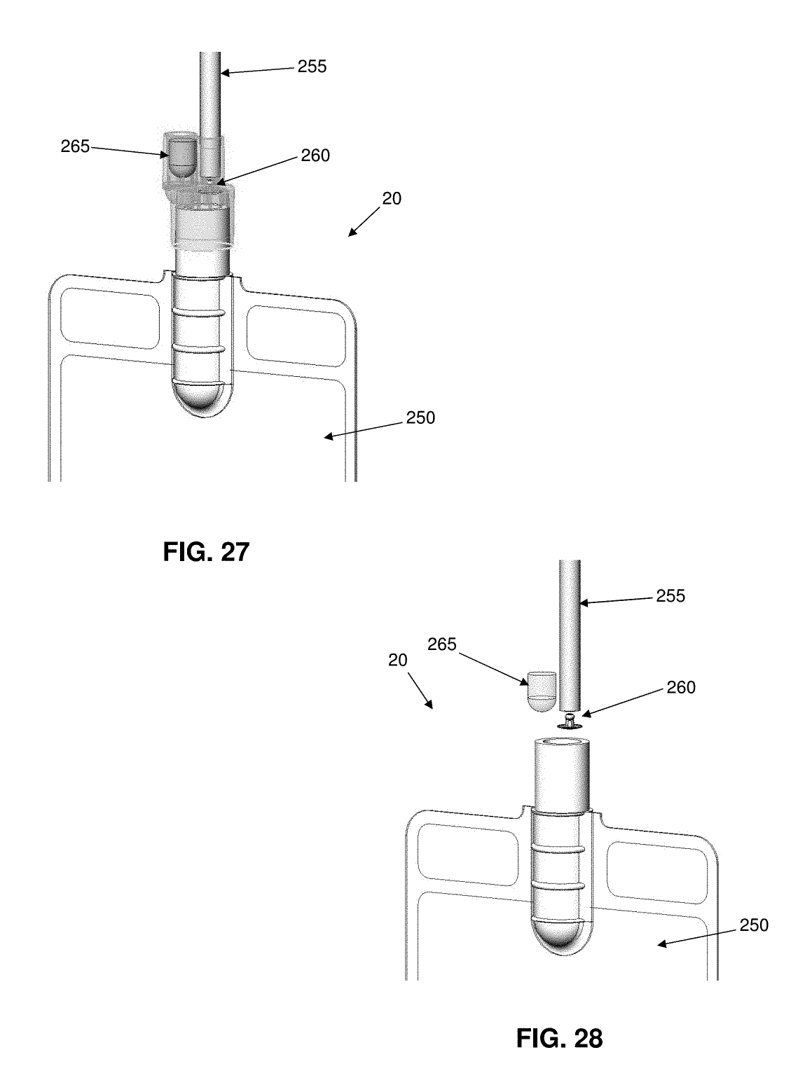

More particularly, in one preferred form of the invention, and looking now at FIGS. 26-28, disposable collection bag 20 comprises a bladder 250 connected to purge port 45 of disposable cartridge 10 via a fluid line 255. A one-way valve 260 is disposed between fluid line 255 and bladder 250 so as to ensure that fluid may flow from fluid line 255 into bladder 250 but fluid is prevented from flowing from bladder 250 back into fluid line 255. A flow inhibitor 265 connects the interior of bladder 250 with the atmosphere, such that air (e.g., from detected gas masses and from accumulated microbubbles purged with detected gas masses) may flow from the interior of bladder 250 to the atmosphere but liquid in bladder 250 is inhibited from flowing out of bladder 250.

### Use

One preferred method of using system 5 will now be described.

First, base unit 15 of system 5 is disposed vertically (e.g., with a disposition such as is shown in FIG. 1) adjacent to a patient (e.g., base unit 15 is appropriately mounted to an infusion line pole), and then base unit 5 is turned on.

Next, a fresh disposable cartridge 10 is removed from its sterilized packaging (preferably with disposable collection bag 20 already connected to purge port 45) and removable mechanical stop 125 of disposable cartridge 10 is pulled free from body 25, allowing resilient valve element 110 to close off third pinch valve section 105 of third passageway 80. Then disposable cartridge 10 is loaded onto door 150 of base unit 15, and snapped into place, so that disposable cartridge 10 is held to door 150 by means of (i) first door edge 220 and its associated flange 221, second door edge 222, and third door edge 223 and its associated flange 224, and (ii) spring tab 48 on disposable cartridge 10. When disposable cartridge 10 is mounted on door 150 of base unit 15 in this manner, first finger 230 of door 150 engages first bubble detection section 55 of disposable cartridge 10, second finger 235 of door 150 engages second bubble detection section 85 of disposable cartridge 10, and third finger 240 of door 150 engages second end 120 of resilient valve element 110 and forces second end 120 of resilient valve element 110 away from door 150, whereby to reopen third pinch valve section 105 of third passageway 80 of disposable cartridge 10.

Then door 150 is closed and latched shut, i.e., by means of latch finger 245 of door 150 engaging latch seat 157 of housing 145. When door 150 is closed and latched shut:
(i) protrusion 140 of disposable cartridge 10 engages cartridge sensor 160 on housing 145, whereby to advise CPU 210 that a disposable cartridge 10 is appropriately mounted to base unit 15;
(ii) first bubble detection section 55 of disposable cartridge 10 is seated in first ultrasound bubble detector 165 on housing 145, with first finger 230 of door 150 ensuring that first bubble detection section 55 is securely seated in first channel 167 of first ultrasound bubble detector 165, with first ultrasound transducer 168 disposed adjacent to first bubble detection section 55, such that first ultrasound bubble detector 165 can advise CPU 210 when a gas bubble is disposed in first bubble detection section 55 of disposable cartridge 10;
(iii) second bubble detection section 85 of disposable cartridge 10 is seated in second ultrasound bubble detector 170 on housing 145, with second finger 235 of door 150 ensuring that second bubble detection section 85 is securely seated in second channel 172 of second ultrasound bubble detector 170, with second ultrasound transducer 173 disposed adjacent to second bubble detection section 85, such that second ultrasound bubble detector 170 can advise CPU 210 when a gas bubble is disposed in second bubble detection section 85 of disposable cartridge 10;
(iv) first pinch valve head 180 of first solenoid 175 is aligned with first pinch valve section 70 of disposable cartridge 10 and, since first solenoid 175 is a so-called “spring-return” solenoid and first solenoid 175 is disposed in housing 145 such that first pinch valve head 180 is normally biased inwardly (i.e., away from a disposable cartridge mounted in door 150), first solenoid 175 causes first pinch valve head 180 to be withdrawn from first pinch valve section 70, whereby to not collapse first pinch valve section 70 between first pinch valve head 180 and protrusion 78 of second opening 75 of body 25; and
(v) second pinch valve head 190 of second solenoid 185 is aligned with second pinch valve section 95 of disposable cartridge 10 and, since second solenoid 185 is a so-called “spring-return” solenoid and second solenoid 185 is disposed in housing 145 such that second pinch valve head 190 is normally biased outwardly (i.e., toward a disposable cartridge 10 mounted in door 150), second solenoid 185 causes second pinch valve head 190 to be advanced toward second pinch valve section 95, whereby to collapse second pinch valve section 95 between second pinch valve head 190 and protrusion 102 of fourth opening 100 of body 25.

When door 150 is closed, cartridge sensor 160 is engaged by protrusion 140 of disposable cartridge 10, so that cartridge sensor 160 (and hence CPU 210) knows that a disposable cartridge 10 is appropriately mounted to door 150 and that door 150 is closed. CPU 210 polls first ultrasound bubble detector 165 to determine if an air bubble is disposed in first bubble detection section 55, and polls second ultrasound bubble detector 170 to determine if an air bubble is disposed at second bubble detection section 85. Since disposable cartridge 10 has not yet been connected to an infusion line, there is only air in disposable cartridge 10, and first ultrasound bubble detector 165 detects air in first bubble detection section 55 and second ultrasound bubble detector 170 detects air in second bubble detection section 85. CPU 210 therefore actuates first solenoid 175, driving its first pinch valve head 180 against first pinch valve section 70, whereby to close off first pinch valve section 70 to flow, and CPU 210 actuates second solenoid 185, withdrawing its second pinch valve head 190 from second pinch valve section 95, whereby to open second pinch valve section 95 to flow. In other words, CPU 210 causes solenoid 175 to close off second passageway 65 of disposable cartridge 10 to fluid flow at first pinch valve section 70, and CPU 210 causes solenoid 185 to open up third passageway 80 of disposable cartridge 10 to fluid flow at second pinch valve section 95. Thus, at this point, inlet port 30 of disposable cartridge 10 is connected to purge port 45 of disposable cartridge 10 and inlet port 30 of disposable cartridge 10 is “disconnected” from (i.e., is not in fluid communication with) outlet port 40 of disposable cartridge 10. In other words, at this point, the supply side of the infusion line is connected to disposable collection bag 20 through disposable cartridge 10.

Then inlet port 30 is connected to the supply side of an infusion line, and outlet port 40 is connected to the patient side of an infusion line. Purge port 45 is connected to disposable collection bag 20 via fluid line 255.

As liquid starts to flow through first passageway 50 of disposable cartridge 10, first ultrasound bubble detector 165 detects liquid at first bubble detection section 55, but second ultrasound bubble detector 170 detects air at second bubble detection section 85. Therefore, CPU 210 maintains first solenoid 175 and second solenoid 185 in their current dispositions.

Liquid flows through first passageway 50 of disposable cartridge 10 and into chamber 35 of disposable cartridge 10 and fills second passageway 65 of disposable cartridge 10 down to first pinch valve section 70, which is blocked off by the engagement of first pinch valve head 180 with first pinch valve section 70. Continued fluid flow fills chamber 35 of disposable cartridge 10, and then fills third passageway 80 of disposable cartridge 10 until liquid exits purge port 45 and enters fluid line 255 leading to disposable collection bag 20.

At this point, first ultrasound bubble detector 165 detects liquid in first bubble detection section 55 of disposable cartridge 10 and second ultrasound bubble detector 170 detects liquid in bubble detection section 85 of disposable cartridge 10. CPU 210 therefore causes first solenoid 175 to retract its first pinch valve head 180 from first pinch valve section 70, whereby to reopen first pinch valve section 70 to flow, and CPU 210 causes second solenoid 185 to drive its second pinch valve head 190 against second pinch valve section 95, whereby to close second pinch valve section 95 to flow. In other words, CPU 210 uses solenoids 175 and 185 to open up second passageway 65 of disposable cartridge 10 to fluid flow at first pinch valve section 70 and to close down third passageway 80 of disposable cartridge 10 to fluid flow at second pinch valve section 95. Thus, at this point, inlet port 30 of disposable cartridge 10 is connected to outlet port 40 of disposable cartridge 10 and inlet port 30 of disposable cartridge 10 is “disconnected” from (i.e., is not in fluid communication with) purge port 45 of disposable cartridge 10. In other words, at this point, the supply side of the infusion line is connected to the patient side of the infusion line through disposable cartridge 10.

The infusion liquid is passed down the patient side of the infusion line until the patient side of the infusion line is completely filled with liquid, and then the patient side of the infusion line may be connected to the patient.

Note that any microbubbles passing into disposable cartridge 10 which are too small for detection by first ultrasound bubble detector 165 of base unit 15 will tend to collect at the top of chamber 35 and then pass into the adjacent, uppermost portions of third passageway 80, thereby preventing microbubbles from passing to the patient. These “collected” microbubbles remain at the uppermost portions of third passageway 80 and are purged when a gas bubble is thereafter purged from the system.

When a gas bubble large enough to be detected by system 5 is detected at first bubble detection section 55, CPU 210 causes first solenoid 175 to drive its first pinch valve head 180 against first pinch valve section 70, whereby to close off first pinch valve section 70 to flow, and CPU 210 causes second solenoid 185 to retract its second pinch valve head 190 from second pinch valve section 95, whereby to open second pinch valve section 95 to flow. In other words, CPU 210 uses solenoids 175 and 185 to close off second passageway 65 of disposable cartridge 10 to fluid flow at first pinch valve section 70 and to open up third passageway 80 of disposable cartridge 10 to fluid flow at second pinch valve section 95. Thus, at this point, inlet port 30 of disposable cartridge 10 is connected to purge port 45 of disposable cartridge 10 and inlet port 30 of disposable cartridge 10 is “disconnected” from (i.e., is not in fluid communication with) outlet port 40 of disposable cartridge 10. In other words, at this point, the supply side of the infusion line is connected to disposable collection bag 20 through disposable cartridge 10. This causes the fluid in disposable cartridge 10 to be diverted to purge port 45, carrying with it the gas bubble detected at first bubble detection section 55.

This purging action continues until first ultrasound bubble detector 165 is detecting liquid in first bubble detection section 55 and second ultrasound bubble detector 170 is detecting liquid in second bubble detection section 85 (note that where the gas bubble is long enough to extend between first ultrasound bubble detector 165 and second ultrasound bubble detector 170, the gas bubble will be completely purged before first ultrasound bubble detector 165 and second ultrasound bubble detector 170 both detect liquid again; note also that where the gas bubble is not long enough to extend between first ultrasound bubble detector 165 and second ultrasound bubble detector 170, the purging stops after the gas bubble clears first ultrasound bubble detector 165 but before the gas bubble reaches second ultrasound bubble detector 170, since first ultrasound bubble detector 165 and second ultrasound bubble detector 170 again both detect liquid—in this case, the gas bubble remains in the line, e.g., stored in third passageway 80, etc., waiting until purging commences again. Once first ultrasound bubble detector 165 is detecting liquid in first bubble detection section 55 and second ultrasound bubble detector 170 is detecting liquid in second bubble detection section 85, CPU 210 then causes first solenoid 175 to retract its first pinch valve head 180 from first pinch valve section 70, whereby to reopen first pinch valve section 70 to flow, and CPU 210 causes second solenoid 185 to drive its second pinch valve head 190 against second pinch valve section 95, whereby to close second pinch valve section 95 to flow. In other words, CPU 210 uses solenoids 175 and 185 to open up second passageway 65 of disposable cartridge 10 to fluid flow at first pinch valve section 70 and to close down third passageway 80 of disposable cartridge 10 at second pinch valve section 95. Thus, at this point, inlet port 30 of disposable cartridge 10 is connected to outlet port 40 of disposable cartridge 10 and inlet port 30 of disposable cartridge 10 is “disconnected” from (i.e., is not in fluid communication with) purge port 45 of disposable cartridge 10. In other words, at this point, the supply side of the infusion line is once again connected to the patient side of the infusion line through disposable cartridge 10.

The infusion flow continues, with the infusion fluid flowing from the fluid source, into inlet port 30, through first passageway 50, into chamber 35, past first pinch valve section 70, out outlet port 40, and on to the patient. As the fluid flows through disposable cartridge 10, first ultrasound bubble detector 165 monitors the fluid flow, sensing for the presence of a gas bubble. So long as no gas bubble is detected by first ultrasound bubble detector 165, the fluid is allowed to flow uninterrupted, thereby infusing the patient with the desired fluid. Any microbubbles entrained in the infusion fluid are collected at the top end of chamber 35 and then pass into the adjacent, uppermost portions of third passageway 80, effectively being stored for purging with the next gas bubble to be detected and purged. In any case, the microbubbles do not pass to the patient. In the event that first ultrasound bubble detector 165 detects a gas bubble (e.g., an air bubble) in the infusion fluid, CPU 210 closes off first pinch valve section 70 in second passageway 65, thereby closing off flow to the patient, and opens second pinch valve section 95 in third passageway 80, thereby allowing fluid to flow from chamber 35, through third passageway 80 and out purge port 45 to disposable collection bag 20. The infusion fluid is allowed to flow out purge port 45 and into disposable collection bag 20 until liquid is again detected by both first ultrasound bubble detector 165 and second ultrasound bubble detector 170, whereupon CPU 210 closes off second pinch valve section 95 in third passageway 80, terminating fluid flow out purge port 45, and opens first pinch valve section 70 in second passageway 65, thereby restoring fluid flow to the patient.

In this way, system 5 automatically detects and removes gas masses from the infusion fluid, thereby preventing the gas masses from being introduced into the patient. In addition, due to the configuration of disposable cartridge 10, system 5 also aggregates and purges microbubbles entrained in the infusion fluid.

In the event that system 5 detects a malfunction, or in the event that tilt sensor 192 detects that system 5 has been knocked over, display 195 and/or audio speaker 500 may be used to advise the user.

Note that if the user opens door 150 of base unit 15 and releases disposable cartridge 10 from door 150, third finger 240 on door 150 will no longer bear against second end 120 of resilient valve element 110, so that resilient valve element 110 will automatically close off third pinch valve section 105 of third passageway 80 to fluid flow, thereby ensuring that all fluid is directed to the patient.

### High System Flow Rates

Significantly, the architecture of disposable cartridge 10 allows system 5 to be run at unusually high flow rates (e.g., 1200 mL/minute) without a concern of entraining air in the infusion line within disposable cartridge 10.

More particularly, after disposable cartridge 10 has been “primed” and is ready for infusion of the patient to begin, there is no air within the disposable cartridge—first passageway 50, chamber 35, second passageway 65 and third passageway 80 are all completely filled with liquid. Inasmuch as there is no air/liquid interface within the disposable cartridge, there is little concern about entraining air in the liquid as the liquid flows through the cartridge.

Furthermore, by connecting first passageway 50 to the side wall of chamber 35 near the top end of chamber 35, with first passageway 50 entering the side wall of chamber 35 with an upward incline, i.e., so that passageway 50 is “pointing upwards”, towards the top end of chamber 35, any gas masses contained in the infusion fluid, and any microbubbles entrained in the infusion fluid, are already oriented toward the top end of chamber 35, thereby facilitating passage of the gas masses and entrained microbubbles out of the top end of chamber 35 and into the adjacent uppermost portions of third passageway 80. Note that this keeps the top end of chamber 35 from accumulating gas, whereby to eliminate an air-liquid interface at the top end of chamber 35.

### Calculation Of Gas Bubble Volume

In some situations it can be useful to determine, and record/report, the volume of a gas bubble in the infusion line. This can be done by determining the time at which the leading end of a gas bolus passes by first ultrasound bubble detector 165, and by determining the time at which the trailing end of the gas bolus passes by first ultrasound bubble detector 165, whereby to determine the length of time it takes for the entire gas bolus to pass by first ultrasound bubble detector 165. Then, inasmuch as the flow rate in first bubble detection section 55 is known, and inasmuch as the internal cross-sectional area of first bubble detection section 55 is known, it is possible to compute the volume of the gas bubble passing by first ultrasound bubble detector 165. This computed volume can be recorded/reported by CPU 210.

Note that if the flow rate in first bubble detection section 55 is not known, it can be calculated by determining the length of time that it takes the gas bolus to pass from first ultrasound bubble detector 165 to second ultrasound bubble detector 170 (since the distance of the flow path between first ultrasound bubble detector 165 to second ultrasound bubble detector 170 is known).

### Alternative Ultrasound Bubble Detectors

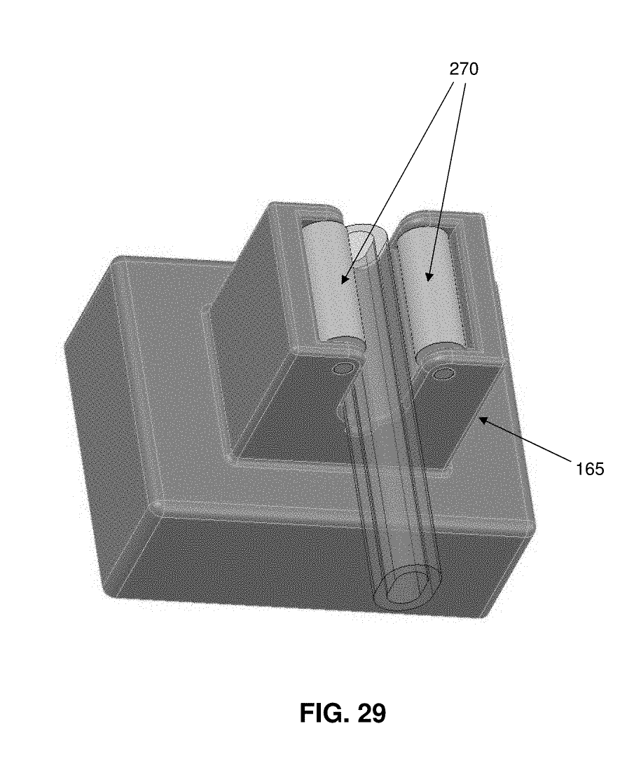

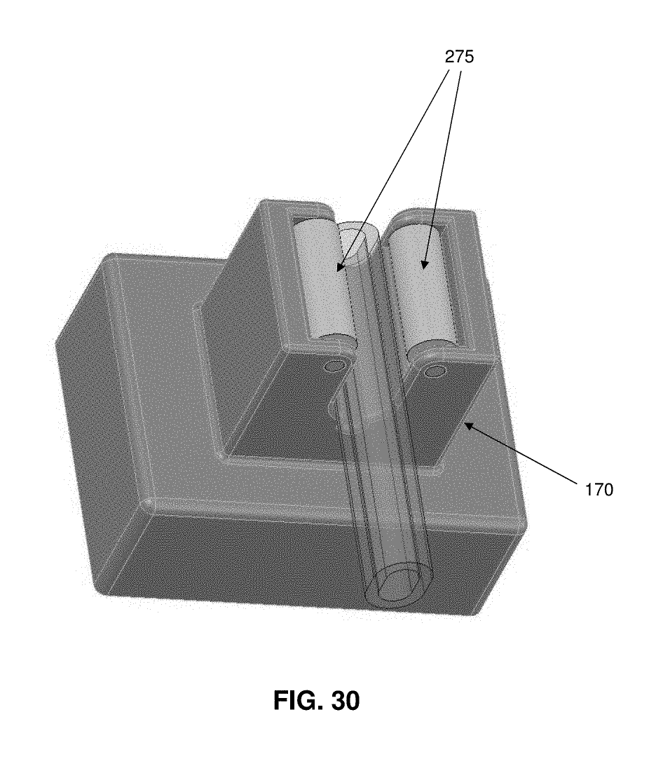

If desired, and looking now at FIG. 29, first ultrasound bubble detector 165 can be provided with rollers 270 at the entrances to first channel 167. Rollers 270 can facilitate insertion of first bubble detection section 55 into first channel 167. Additionally and/or alternatively, and looking now at FIG. 30, second ultrasound bubble detector 170 can be provided with rollers 275 at the entrances to second channel 172. Rollers 275 can facilitate insertion of second bubble detection section 85 into second channel 172.

### Alternative Finger Profiles

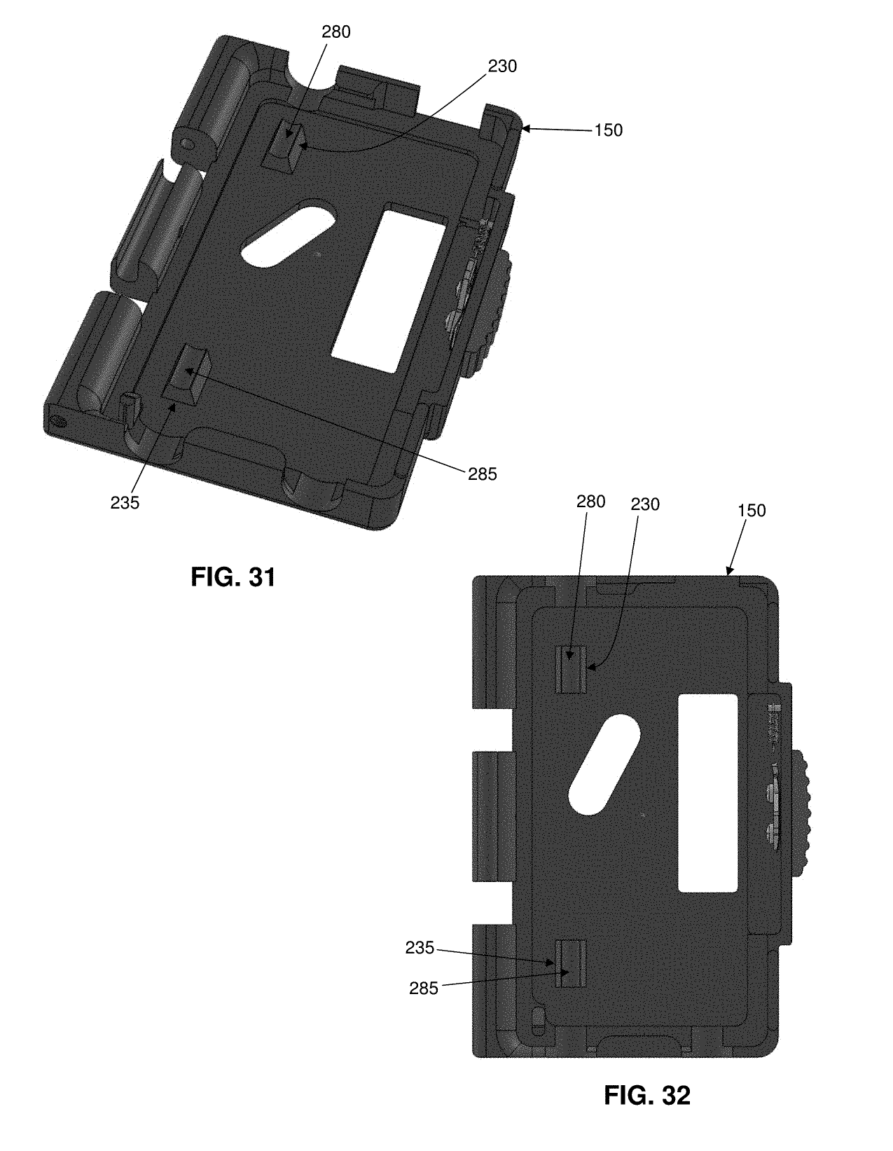

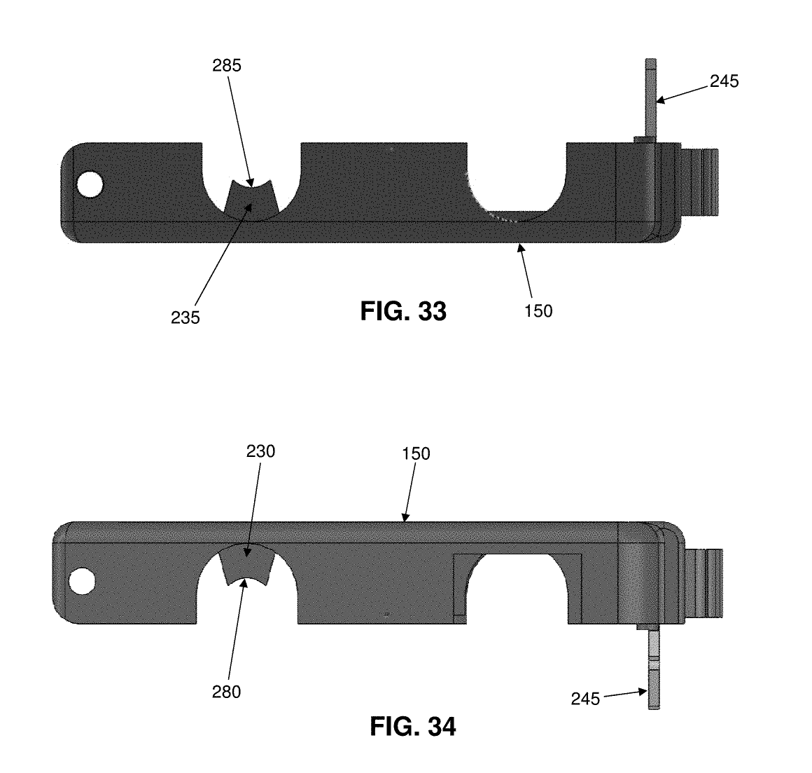

If desired, and looking now at FIGS. 31-34, first finger 230 of door 150 can be formed with a concave profile as shown at 280 to facilitate proper positioning of first bubble detection section 55 of disposable cartridge 10 in first channel 167 of first ultrasound bubble detector 165 of base unit 15, and/or second finger 235 of door 150 can be formed with a concave profile as shown at 285 to facilitate proper positioning of second bubble detection section 85 of disposable cartridge 10 in second channel 172 of second ultrasound bubble detector 170 of base unit 15.

More particularly, forming first finger 230 with a concave profile helps the tubing of first bubble detection section 55 to better seat in first channel 167 of first ultrasound bubble detector 165, whereby to provide improved acoustic coupling for air/liquid detection. In addition, the curvature of concavity 280 relieves pressure on the center of the tube, and compresses the tube slightly on the two sides of the tube as the tube slides into first channel 167. This helps prevent the tube from spreading laterally as it gets compressed and helps prevent possible deformation of the tube.

In a similar manner, forming second finger 235 with a concave profile helps the tubing of second bubble detection section 85 to better seat in second channel 172 of second ultrasound bubble detector 170, whereby to provide improved acoustic coupling for air/liquid detection. In addition, the curvature of concavity 285 relieves pressure on the center of the tube, and compresses the tube slightly on the two sides of the tube as the tube slides into second channel 172. This helps prevent the tube from spreading laterally as it gets compressed and helps prevent possible deformation of the tube.

### Alternative Approach for Mounting the Disposable Cartridge on the Door

In the foregoing description, door 150 is described as being configured to releasably mount disposable cartridge 10 thereon via (i) first door edge 220 and its associated flange 221, a second door edge 222, and a third door edge 223 and its associated flange 224, and (ii) spring tab 48 on disposable cartridge 10. And in the foregoing description, disposable cartridge 10 is described as being releasably mounted to door 150 by positioning spring tab 48 of disposable cartridge 10 on third door edge 223, behind its associated flange 224; pushing downward on disposable cartridge 10 so as to compress spring tab 48 on disposable cartridge 10; pushing the top end of disposable cartridge 10 toward door 150 so that the top end of disposable cartridge 10 clears flange 221 on first door edge 220; and then releasing disposable cartridge 10 so that spring tab 48 on disposable cartridge 10 pushes the top end of disposable cartridge 10 up behind flange 221 of first door edge 220. Thus, loading of disposable cartridge 10 on door 150 is something of a “bottom up” process.

However, other arrangements may be provided for releasably mounting disposable cartridge 10 on door 150.

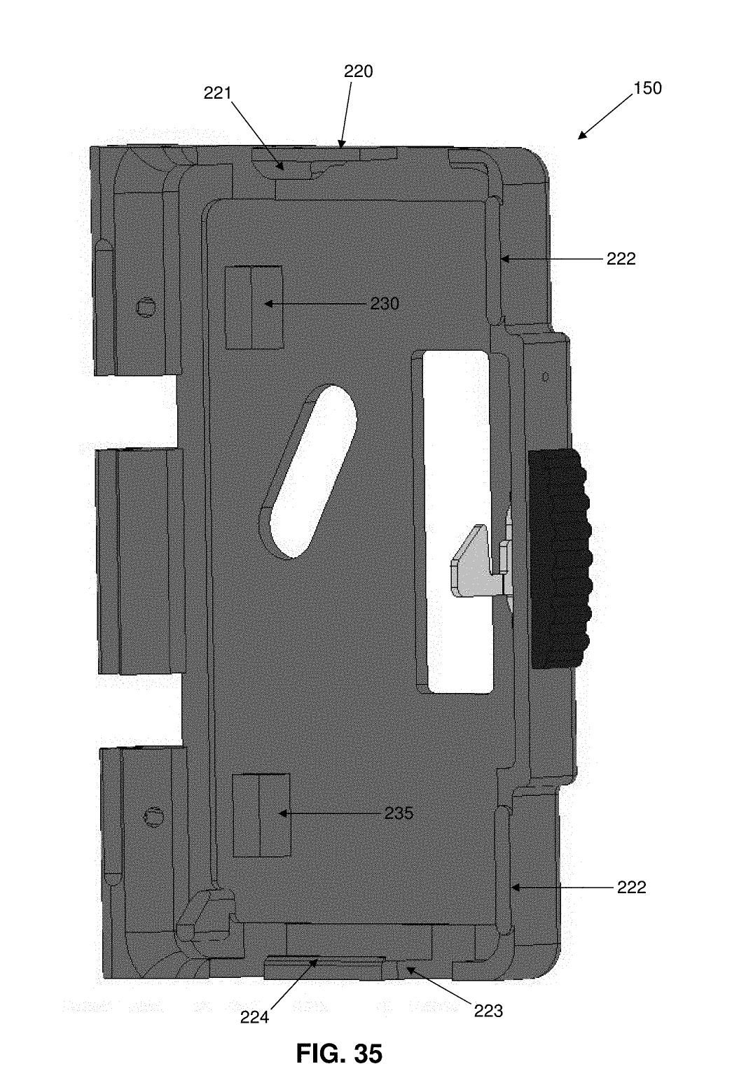

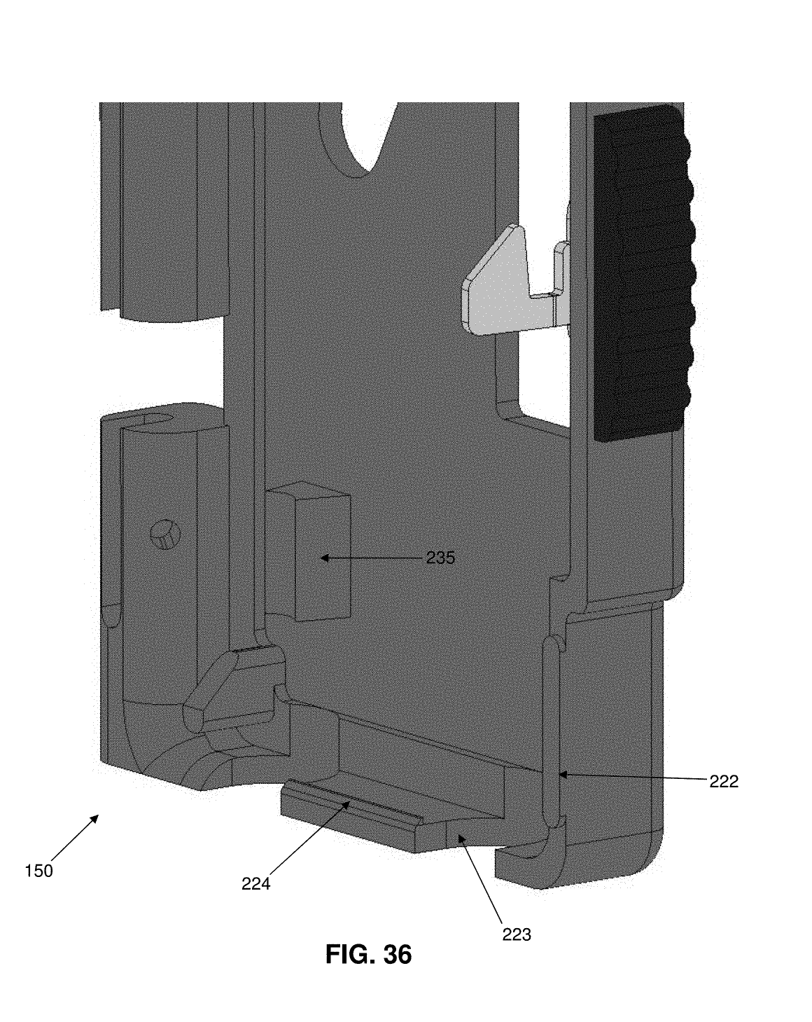

By way of example but not limitation, and looking now at FIGS. 35 and 36, associated flange 224 of third door edge 223 may have a reduced height and a ramp/chamfer lead-in, such that disposable cartridge 10 may be releasably mounted to door 150 by positioning the top end of disposable cartridge 10 behind associated flange 221 of fixed tab 220, and then pushing the bottom of disposable cartridge 10 towards the door so that spring tab 48 on disposable cartridge 10 pushes past the ramp/chamfer lead-in of associated flange 224 of third door edge 223 and snaps into place. Thus, with this form of the invention, loading of disposable cartridge 10 on door 150 is something of a “top down” process. Significantly, this arrangement produces an audible click as the bottom end of disposable cartridge 10 snaps into place, thereby providing useful feedback for the user.

### Alternative Tube Holder

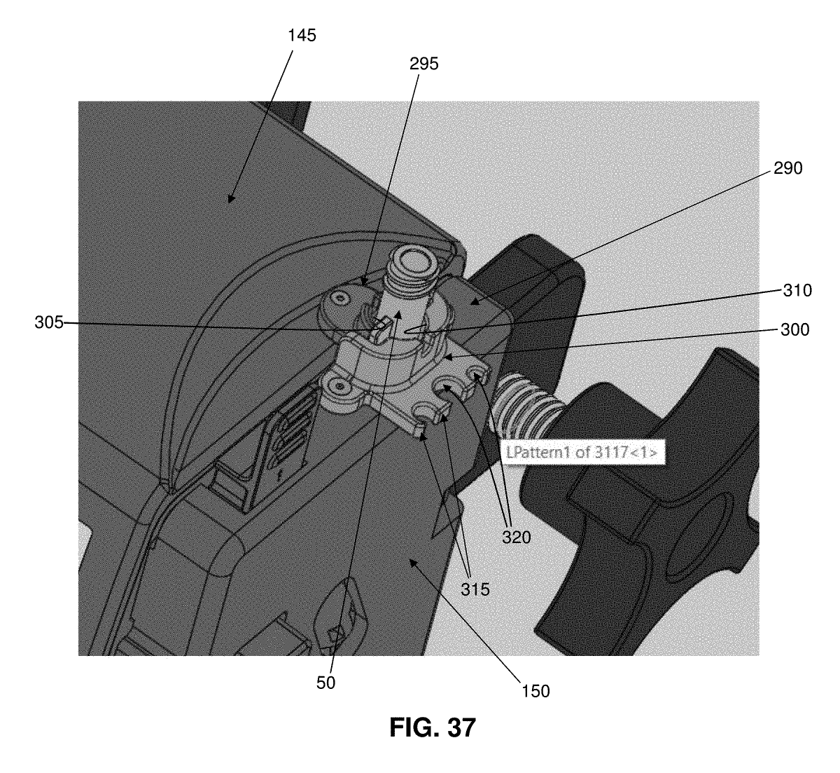

In another form of the invention, tube holder/hook 247 may be replaced by an alternative apparatus. More particularly, in this form of the invention, and looking now at FIG. 37, system 5 may comprise a tube holder 290. Tube holder 290 generally comprises a first portion 295 mounted to housing 145 of base unit 15, and a second portion 300 mounted to door 150 of base unit 15. First portion 295 comprises a first concave seat 305, and second portion 300 comprises a second concave seat 310, with first concave seat 305 and second concave seat 310 cooperating with one another so as to form a substantially cylindrical recess for receiving and supporting the upper portion of first passageway 50 (i.e., the portion of first passageway 50 adjacent to inlet port 30) when door 150 is closed against housing 145. And in this form of the invention, second portion 300 of tube holder 290 comprises one or more fingers 315 which together form one or more seats 320 for receiving and supporting excess infusion line tubing adjacent to base unit 15.

### Alternative Disposable Collection Bag

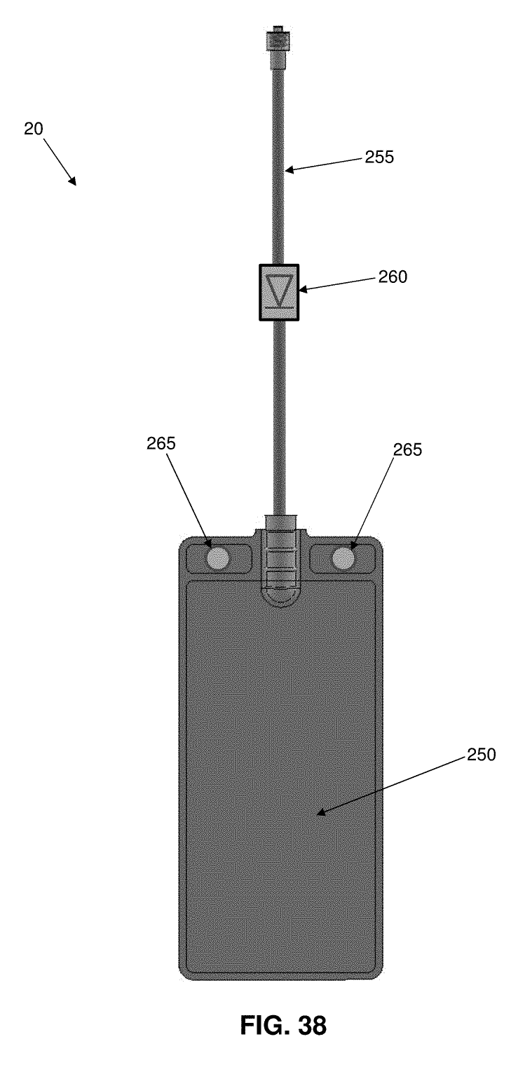

If desired, and looking now at FIG. 38, disposable collection bag 20 may be modified so as to vary the positions of one-way valve 260 and/or flow inhibitor(s) 265 from that shown previously.

More particularly, in this alternative form of the invention, one-way valve 260 may be moved upward along fluid line 255 so as to further space one-way valve 260 from bladder 250. This construction has the advantage of reducing turbulence and splashing at the inlet to bladder 250, particularly at high flow rates.

And flow inhibitor 265 may be moved to one or more locations at the top of bladder 250, away from the inlet to bladder 250, such as is shown in FIG. 38. This construction has the advantage of reducing turbulence and splashing at restrictor(s) 265 which could wet and saturate restrictor(s) 265, particularly at high flow rates.

### Elimination Of Resilient Valve Element 110

In the foregoing description, disposable cartridge 10 is described as comprising a resilient valve element 110 for selectively closing off third pinch valve section 105, e.g., in the event of system failure, door 150 can be manually opened and disposable cartridge 10 manually dismounted from door 150, whereupon resilient valve element 110 automatically blocks third pinch valve section 105, whereby to direct all fluid flow to the patient.

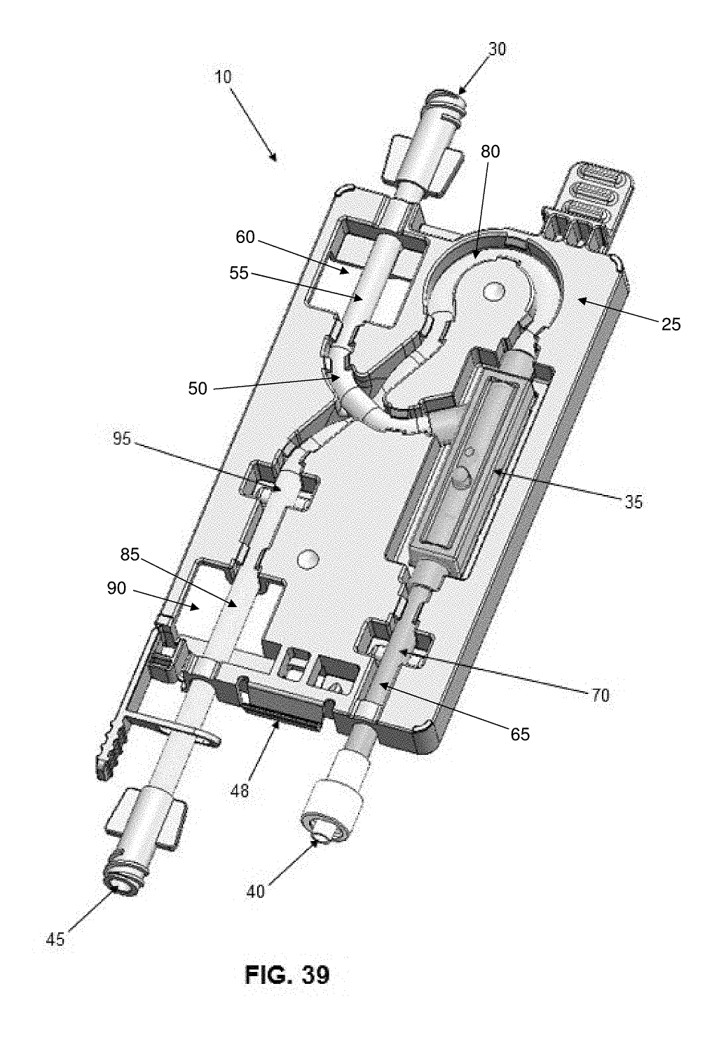

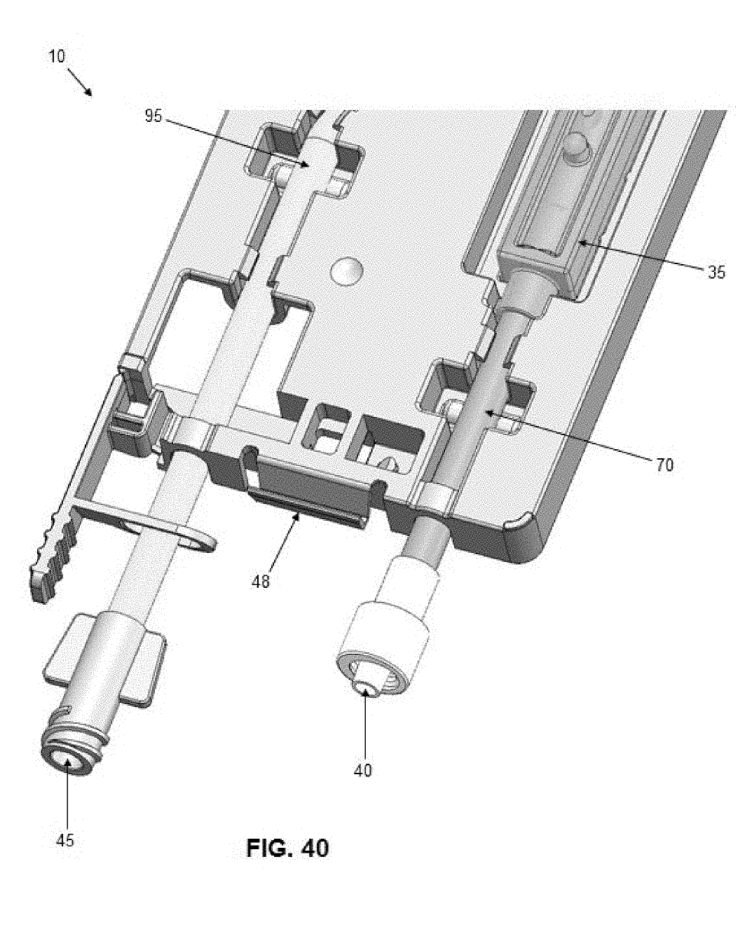

However, if desired, and looking now at FIGS. 39 and 40, resilient valve element 110 may be eliminated from disposable cartridge 150. Thus, in this form of the invention, there is no resilient element for automatically closing off third pinch valve section 105 of disposable cartridge 10 in the event of system failure. Instead, in this form of the invention, CPU 210 is programmed so that in the event of system failure, first solenoid 175 moves first pinch valve head 180 away from first pinch valve section 70, and second solenoid 185 moves second pinch valve head 190 into engagement with second pinch valve section 95, whereby to direct all fluid flow to the patient.

### Modifications

It will be appreciated that further embodiments of the present invention will be apparent to those skilled in the art in view of the present disclosure.

It is to be understood that the present invention is by no means limited to the particular constructions herein disclosed and/or shown in the drawings, but also comprises any modifications or equivalents within the scope of the invention.

What is claimed is:

1. Apparatus for detecting and removing a gas mass from a vascular infusion line, said apparatus comprising:
a cartridge, said cartridge comprising:
an inlet port;
a chamber having a top end, a bottom end and a side wall extending therebetween;
an outlet port;
a purge port;
a first passageway connecting said inlet port to said side wall of said chamber with an upward inclination;
a second passageway connecting said bottom end of said chamber to said outlet port; and
a third passageway connecting said top end of said chamber to said purge port.

2. Apparatus according to claim 1 wherein said cartridge further comprises a body, and further wherein said inlet port, said chamber, said purge port, said first passageway, said second passageway and said third passageway are mounted to said body.

3. Apparatus according to claim 2 wherein said cartridge further comprises:
a first bubble detection section disposed in said first passageway intermediate said inlet port and said chamber, wherein said first bubble detection section is constructed so as to enable the detection of a bubble within said first bubble detection section;
a second bubble detection section disposed in said third passageway intermediate said chamber and said purge port, wherein said second bubble detection section is constructed so as to enable the detection of a bubble within said second bubble detection section;
a first opening formed in said body for providing access to at least a portion of said first bubble detection section; and
a second opening formed in said body for providing access to at least a portion of said second bubble detection section.

4. Apparatus according to claim 3 wherein said cartridge further comprises:
a first pinch valve section disposed in said second passageway intermediate said chamber and said outlet port, wherein said first pinch valve section is constructed so as to enable selective closure of said first pinch valve section; and
a second pinch valve section disposed in said third passageway intermediate said chamber and said purge port, wherein said second pinch valve section is constructed so as to enable selective closure of said second pinch valve section.

5. Apparatus according to claim 4 wherein said cartridge further comprises:
a third pinch valve section disposed in said third passageway between said second pinch valve section and said purge port, wherein said third pinch valve section is constructed so as to enable selective closure of said third pinch valve section;
a resilient valve element mounted to said body and configured so that in its unbiased state, said resilient valve element will close off said third pinch valve section; and
a removable mechanical stop releasably mounted to said body which, when in position on said body, keeps said resilient valve element from closing off said third pinch valve section.

6. Apparatus according to claim 4, further comprising:
a base unit, said base unit comprising a housing and a door movably mounted to said housing between an open position and a closed position;
wherein said door is configured to releasably seat said cartridge;
wherein said housing comprises a first ultrasound bubble detector and a second ultrasound bubble detector, with said first bubble detection section of said cartridge being aligned with said first ultrasound bubble detector of said housing, and said second bubble detection section of said cartridge being aligned with said second ultrasound bubble detector of said housing, when said cartridge is seated on said door and said door is in said closed position;
wherein said housing comprises a first pinch valve head and a second pinch valve head, with said first pinch valve section of said cartridge being aligned with said first pinch valve head of said housing, and said second pinch valve section of said cartridge being aligned with said second pinch valve head of said housing, when said cartridge is seated on said door and said door is in said closed position.

7. Apparatus according to claim 6 further comprising a first solenoid for moving said first pinch valve head, a second solenoid for moving said second pinch valve head, and a central processing unit for controlling said first solenoid and said second solenoid.

8. Apparatus according to claim 7 further comprising a collection bag connected to said purge port.

9. Apparatus according to claim 7 further comprising a tilt sensor connected to said central processing unit.

10. A method for detecting and removing a gas mass from a vascular infusion line, said method comprising:
providing apparatus comprising:
a cartridge, said cartridge comprising:
an inlet port;
a chamber having a top end, a bottom end and a side wall extending therebetween;
an outlet port;
a purge port;
a first passageway connecting said inlet port to said side wall of said chamber with an upward inclination;
a second passageway connecting said bottom end of said chamber to said outlet port; and
a third passageway connecting said top end of said chamber with said purge port;
closing off said second passageway;
introducing a liquid into said inlet port until said liquid exits said purge port;
closing off said third passageway; and
opening up said second passageway so that liquid exits said outlet port.

11. A method according to claim 10 further comprising:
detecting when a gas mass is disposed in said first passageway, and in response thereto, closing off said second passageway and opening said third passageway; and
detecting when liquid is disposed in said first passageway and said third passageway, and in response thereto, closing off said third passageway and opening said second passageway.

12. Apparatus for detecting and removing a gas mass from a vascular infusion line, said apparatus comprising:
a cartridge, said cartridge comprising:
an inlet port;
a chamber;
an outlet port;
a purge port;
a first passageway connecting said inlet port to said chamber;
a second passageway connecting said chamber to said outlet port; and
a third passageway connecting said chamber to said purge port;
a base unit, said base unit comprising a housing and a door movably mounted to said housing between an open position and a closed position, wherein said door is configured to releasably seat said cartridge; and
at least one projection disposed on said door, said at least one projection comprising a concave surface for engaging at least one of said first passageway and said second passageway when a cartridge is seated on said door and said door is in its closed position.

13. Apparatus according to claim 12 wherein a first projection is disposed on said door, said first projection comprising a concave surface for engaging said first passageway when a cartridge is seated on said door and said door is in its closed position, and wherein a second projection is disposed on said door, said second projection comprising a concave surface for engaging said second passageway when a cartridge is seated on said door and said door is in its closed position.

14. Apparatus according to claim 13 wherein said chamber has a top end, a bottom end, and a side wall extending therebetween, said first passageway connects said inlet port to said side wall of said chamber with an upward inclination, said second passageway connects said bottom end of said chamber to said outlet port, and said third passageway connects said top end of said chamber to said purge port.

15. Apparatus according to claim 14 wherein said cartridge further comprises a body, and further wherein said inlet port, said chamber, said purge port, said first passageway, said second passageway and said third passageway are mounted to said body.

16. Apparatus according to claim 15 wherein said cartridge further comprises:
a first bubble detection section disposed in said first passageway intermediate said inlet port and said chamber, wherein said first bubble detection section is constructed so as to enable the detection of a bubble within said first bubble detection section;
a second bubble detection section disposed in said third passageway intermediate said chamber and said purge port, wherein said second bubble detection section is constructed so as to enable the detection of a bubble within said second bubble detection section;
a first opening formed in said body for providing access to at least a portion of said first bubble detection section; and
a second opening formed in said body for providing access to at least a portion of said second bubble detection section.

17. Apparatus according to claim 16 wherein said cartridge further comprises:
a first pinch valve section disposed in said second passageway intermediate said chamber and said outlet port, wherein said first pinch valve section is constructed so as to enable selective closure of said first pinch valve section; and
a second pinch valve section disposed in said third passageway intermediate said chamber and said purge port, wherein said second pinch valve section is constructed so as to enable selective closure of said second pinch valve section.

18. Apparatus according to claim 17 wherein said cartridge further comprises:
a third pinch valve section disposed in said third passageway between said second pinch valve section and said purge port, wherein said third pinch valve section is constructed so as to enable selective closure of said third pinch valve section;
a resilient valve element mounted to said body and configured so that in its unbiased state, said resilient valve element will close off said third pinch valve section; and
a removable mechanical stop releasably mounted to said body which, when in position on said body, keeps said resilient valve element from closing off said third pinch valve section.

19. Apparatus according to claim 17 wherein said housing comprises a first ultrasound bubble detector and a second ultrasound bubble detector, with said first bubble detection section of said cartridge being aligned with said first ultrasound bubble detector of said housing, and said second bubble detection section of said cartridge being aligned with said second ultrasound bubble detector of said housing, when said cartridge is seated on said door and said door is in said closed position; and
wherein said housing comprises a first pinch valve head and a second pinch valve head, with said first pinch valve section of said cartridge being aligned with said first pinch valve head of said housing, and said second pinch valve section of said cartridge being aligned with said second pinch valve head of said housing, when said cartridge is seated on said door and said door is in said closed position.

20. Apparatus according to claim 19 further comprising a first solenoid for moving said first pinch valve head, a second solenoid for moving said second pinch valve head, and a central processing unit for controlling said first solenoid and said second solenoid.

21. Apparatus according to claim 20 further comprising a collection bag connected to said purge port.

22. Apparatus according to claim 20 further comprising a tilt sensor connected to said central processing unit.

23. Apparatus for detecting and removing a gas mass from a vascular infusion line, said apparatus comprising:
a housing;
a first ultrasound bubble detector mounted to said housing; and
a second ultrasound bubble detector mounted to said housing;
said first ultrasound bubble detector comprising a first channel having an upper end and a lower end, and a first ultrasound transducer disposed adjacent the lower end of said first channel; and
said second ultrasound bubble detector comprising a second channel having an upper end and a lower end, and a second ultrasound transducer disposed adjacent the lower end of said second channel.

24. Apparatus according to claim 23 further comprising a door movably mounted to said housing between an open position and a closed position.

25. Apparatus according to claim 24 further comprising:
a cartridge for seating on said door, said cartridge comprising:
an inlet port;
a chamber having a top end, a bottom end and a side wall extending therebetween;
an outlet port;
a purge port;
a first passageway connecting said inlet port to said side wall of said chamber with an upward inclination;
a second passageway connecting said bottom end of said chamber to said outlet port; and
a third passageway connecting said top end of said chamber to said purge port.

26. Apparatus according to claim 25 wherein said cartridge further comprises a body, and further wherein said inlet port, said chamber, said purge port, said first passageway, said second passageway and said third passageway are mounted to said body.

27. Apparatus according to claim 26 wherein said cartridge further comprises:
a first bubble detection section disposed in said first passageway intermediate said inlet port and said chamber, wherein said first bubble detection section is constructed so as to enable the detection of a bubble within said first bubble detection section;
a second bubble detection section disposed in said third passageway intermediate said chamber and said purge port, wherein said second bubble detection section is constructed so as to enable the detection of a bubble within said second bubble detection section;
a first opening formed in said body for providing access to at least a portion of said first bubble detection section; and
a second opening formed in said body for providing access to at least a portion of said second bubble detection section.

28. Apparatus according to claim 27 wherein said cartridge further comprises:
a first pinch valve section disposed in said second passageway intermediate said chamber and said outlet port, wherein said first pinch valve section is constructed so as to enable selective closure of said first pinch valve section; and
a second pinch valve section disposed in said third passageway intermediate said chamber and said purge port, wherein said second pinch valve section is constructed so as to enable selective closure of said second pinch valve section.

29. Apparatus according to claim 28 wherein said cartridge further comprises:
a third pinch valve section disposed in said third passageway between said second pinch valve section and said purge port, wherein said third pinch valve section is constructed so as to enable selective closure of said third pinch valve section;
a resilient valve element mounted to said body and configured so that in its unbiased state, said resilient valve element will close off said third pinch valve section; and
a removable mechanical stop releasably mounted to said body which, when in position on said body, keeps said resilient valve element from closing off said third pinch valve section.

30. Apparatus according to claim 28 wherein said housing comprises a first pinch valve head and a second pinch valve head, with said first pinch valve section of said cartridge being aligned with said first pinch valve head of said housing, and said second pinch valve section of said cartridge being aligned with said second pinch valve head of said housing, when said cartridge is seated on said door and said door is in said closed position.

31. Apparatus according to claim 30 further comprising a first solenoid for moving said first pinch valve head, a second solenoid for moving said second pinch valve head, and a central processing unit for controlling said first solenoid and said second solenoid.

32. Apparatus according to claim 31 further comprising a collection bag connected to said purge port.

33. Apparatus according to claim 31 further comprising a tilt sensor connected to said central processing unit.
